(19) United States
(12) Patent Application Publication
(10) Pub. No.: US 2003/0032167 A1
(43) Pub. Date: Feb. 13, 2003

(54) Secreted alpha-helical protein zlmda24

(76) Inventors: Darrell C. Conklin, Seattle, WA (US); Zeren Gao, Redmond, WA (US); Catherine E. Lofton-Day, Brier, WA (US); Theodore E. Whitmore, Redmond, WA (US)

Correspondence Address: Jennifer K. Johnson, J.D., ZymoGenetics, Inc., 1201 Eastlake Avenue East, Seattle, WA, 98102, US

(21) Appl. No.: 10/038,241

(22) Filed: Oct. 19, 2001

Related U.S. Application Data
(60) Provisional application No. 60/242,023, filed on Oct. 20, 2000.

(51) Int. Cl. G01N 33/574; C07H 21/04; C12N 9/64; C12P 21/02; C12N 5/06

(52) U.S. Cl. 435/226; 435/69.1; 435/320.1; 435/325; 536/23.2

(57) ABSTRACT

The present invention relates to zlmda24 polynucleotide and polypeptide molecules. Zlmda24 is a novel cytokine-like polypeptide with an anti-parallel alpha-helical structure. The polypeptides may be used within methods for stimulating the proliferation and/or development of hematopoietic cells in vitro and in vivo. Moreover, zlmda24 polynucleotides, polypeptides and antibodies can be used as a diagnostic to detect the presence of human testis tissue. The present invention also includes methods for producing the protein, uses therefor and antibodies thereto.

### REFERENCE TO RELATED APPLICATIONS

This application is related to Provisional Application 60/194,731, filed on Apr. 5, 2000. This application is also related to Provisional Application 60/222,121, filed on Jul. 28, 2000. Under 35 U.S.C. §119(e)(1), this application claims benefit of said Provisional Applications.

### BACKGROUND OF THE INVENTION

Proteins with a basic structure of four alpha-helical bundles in an anti-parallel conformation include hormones and polypeptide growth factors, which control proliferation and differentiation of cells of multicellular organisms. These diffusable molecules allow cells to communicate with each other and act in concert to form cells and organs, and to repair damaged tissue. Examples of hormones and growth factors include the steroid hormones (e.g. estrogen, testosterone), parathyroid hormone, follicle stimulating hormone, the interleukins, platelet derived growth factor (PDGF), epidermal growth factor (EGF), granulocyte-macrophage colony stimulating factor (GM-CSF), erythropoietin (EPO) and calcitonin.

Hormones and growth factors influence cellular metabolism by binding to receptors. Receptors may be integral membrane proteins that are linked to signaling pathways within the cell, such as second messenger systems. Other classes of receptors are soluble molecules, such as the nuclear receptors or transcription factors.

Cytokines generally stimulate proliferation or differentiation of cells of the hematopoietic lineage or participate in the immune and inflammatory response mechanisms of the body. Examples of cytokines which affect hematopoiesis are erythropoietin (EPO), which stimulates the development of red blood cells; thrombopoietin (TPO), which stimulates development of cells of the megakaryocyte lineage; and granulocyte-colony stimulating factor (G-CSF), which stimulates development of neutrophils. These cytokines are useful in restoring normal blood cell levels in patients suffering from anemia, thrombocytopenia, and neutropenia or receiving chemotherapy for cancer.

The interleukins are a family of cytokines that mediate immunological responses, including inflammation. The interleukins mediate a variety of inflammatory pathologies. Central to an immune response is the T cell, which produce many cytokines and adaptive immunity to antigens. Cytokines produced by the T cell have been classified as type 1 and type 2 (Kelso, A. Immun. Cell Biol. 76:300-317, 1998). Type 1 cytokines include IL-2, IFN-γ, LT-α, and are involved in inflammatory responses, viral immunity, intracellular parasite immunity and allograft rejection. Type 2 cytokines include IL-4, IL-5, IL-6, IL-10 and IL-13, and are involved in humoral responses, helminth immunity and allergic response. Shared cytokines between Type 1 and 2 include IL-3, GM-CSF and TNF-α. There is some evidence to suggest that Type 1 and Type 2 producing T cell populations preferentially migrate into different types of inflamed tissue.

Mature T cells may be activated, i.e., by an antigen or other stimulus, to produce, for example, cytokines, biochemical signaling molecules, or receptors that further influence the fate of the T cell population.

B cells can be activated via receptors on their cell surface including B cell receptor and other accessory molecules to perform accessory cell functions, such as production of cytokines.

Natural killer (NK) cells have a common progenitor cell with T cells and B cells, and play a role in immune surveillance. NK cells, which comprise up 15% of blood lymphocytes, do not express antigen receptors, and therefore do not use MHC recognition as requirement for binding to a target cell. NK cells are involved in the recognition and killing of certain tumor cells and virally infected cells. In vivo, NK cells are believed to require activation, however, in vitro, NK cells have been shown to kill some types of tumor cells without activation.

The demonstrated in vivo activities of the cytokine family illustrates the enormous clinical potential of, and need for, other cytokines, cytokine agonists, and cytokine antagonists. The present invention addresses these needs by providing a new cytokine that stimulates cells of the hematopoietic cell lineage, as well as related compositions and methods.

### BRIEF DESCRIPTION OF THE DRAWINGS

FIG. 1 is a multiple alignment of the mouse zlmda24 polypeptide (mzlmda24) (SEQ ID NO:4), and the human zlmda24 polypeptide (SEQ ID NO:2) of the present invention. The “:” in the figure indicates amino acids that are identical between the mouse and human sequences, and the “.” in the figure indicates amino acids that are conserved substitutions. There is a 69.6% identity between the human and mouse sequences over the entire sequence (253 amino acid overlap).

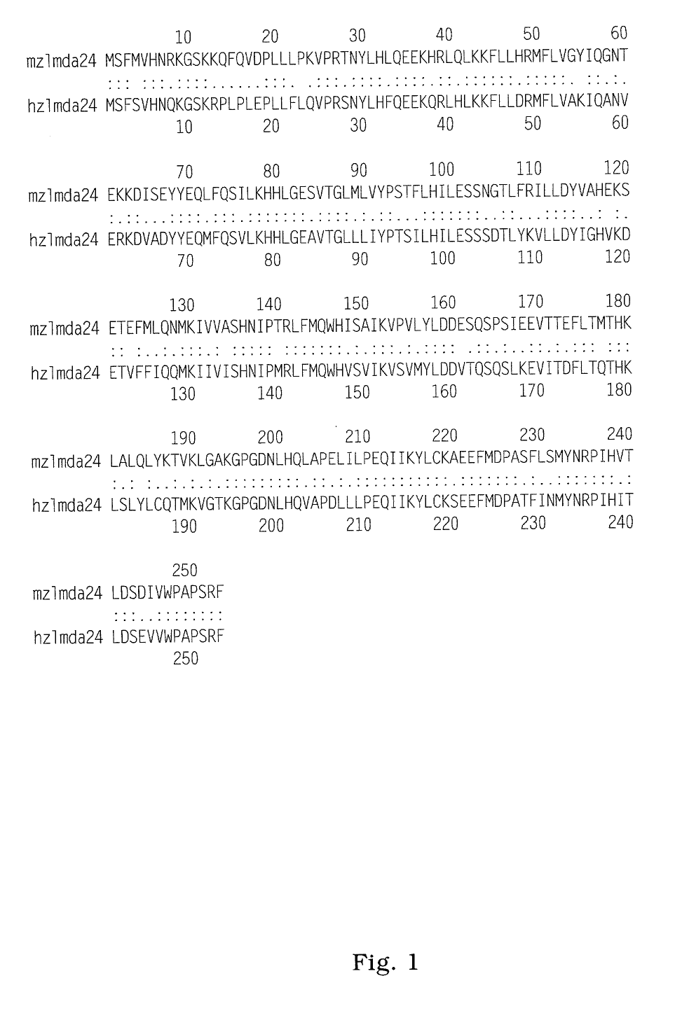

### DESCRIPTION OF THE INVENTION

The present invention provides such polypeptides for these and other uses that should be apparent to those skilled in the art from the teachings herein.

Within one aspect, the present invention provides an isolated polynucleotide that encodes a polypeptide comprising a sequence of amino acid residues that is at least 90% identical to an amino acid sequence selected from the group consisting of: (a) the amino acid sequence as shown in SEQ ID NO:2 from amino acid number 32 (His), to amino acid number 253 (Phe); and (b) the amino acid sequence as shown in SEQ ID NO:2 from amino acid number 1 (Met), to amino acid number 253 (Phe); and (c) a polynucleotide sequence complementary to (a) or (b). In one embodiment, the isolated polynucleotide disclosed above is selected from the group consisting of: (a) a polynucleotide sequence as shown in SEQ ID NO:1 from nucleotide 298 to nucleotide 962; (b) a polynucleotide sequence as shown in SEQ ID NO:1 from nucleotide 205 to nucleotide 962; and (c) a polynucleotide sequence complementary to (a) or (b). In another embodiment, the isolated polynucleotide disclosed above the polynucleotide comprises nucleotide 94 to nucleotide 759 of SEQ ID NO:5. In another embodiment, the isolated polynucleotide disclosed above encodes a polypeptide that comprises a sequence of amino acid residues selected from the group consisting of: (a) the amino acid sequence as shown in SEQ ID NO:2 from amino acid number 32 (His), to amino acid number 253 (Phe); and (b) the amino acid sequence as shown in SEQ ID NO:2 from amino acid number 1 (Met), to amino acid number 253 (Phe); and (c) a polynucleotide sequence complementary to (a) or (b).

Within a second aspect, the present invention provides an expression vector comprising the following operably linked elements: a transcription promoter; a DNA segment encoding a polypeptide as shown in SEQ ID NO:2 from amino acid number 32 (His), to amino acid number 253 (Phe); and a transcription terminator, wherein the promoter is operably linked to the DNA segment, and the DNA segment is operably linked to the transcription terminator. In one embodiment the expression vector disclosed above further comprises a secretory signal sequence operably linked to the DNA segment.

Within a third aspect, the present invention provides a cultured cell comprising an expression vector as disclosed above, wherein the cell expresses a polypeptide encoded by the DNA segment.

Within another aspect, the present invention provides a DNA construct encoding a fusion protein, the DNA construct comprising: a first DNA segment encoding a polypeptide comprising a sequence of amino acid residues selected from the group consisting of: (a) the amino acid sequence as shown in SEQ ID NO:2 from amino acid number 1 (Met), to amino acid number 31 (Leu); (b) the amino acid sequence as shown in SEQ ID NO:4 from amino acid number 1 (Met), to amino acid number 27 (Arg); (c) the amino acid sequence as shown in SEQ ID NO:2 from amino acid number 42 (Leu), to amino acid number 56 (Ile); (d) the amino acid sequence as shown in SEQ ID NO:2 from amino acid number 108 (Tyr), to amino acid number 122 (Thr); (e) the amino acid sequence as shown in SEQ ID NO:2 from amino acid number 151 (Ile), to amino acid number 165 (Gln); (f) the amino acid sequence as shown in SEQ ID NO:2 from amino acid number 213 (Ile), to amino acid number 227 (Ala); (g) the amino acid sequence as shown in SEQ ID NO:2 from amino acid number 42 (Ile), to amino acid number 227 (Ala); (h) the amino acid sequence as shown in SEQ ID NO:2 from amino acid number 32 (His), to amino acid number 253 (Phe); and at least one other DNA segment encoding an additional polypeptide, wherein the first and other DNA segments are connected in-frame; and wherein the first and other DNA segments encode the fusion protein.

Within another aspect, the present invention provides an expression vector comprising the following operably linked elements: a transcription promoter; a DNA construct encoding a fusion protein as disclosed above; and a transcription terminator, wherein the promoter is operably linked to the DNA construct, and the DNA construct is operably linked to the transcription terminator.

Within another aspect, the present invention provides a cultured cell comprising an expression vector as disclosed above, wherein the cell expresses a polypeptide encoded by the DNA construct.

Within another aspect, the present invention provides a method of producing a fusion protein comprising: culturing a cell as disclosed above; and isolating the polypeptide produced by the cell.

Within another aspect, the present invention provides an isolated polypeptide comprising a sequence of amino acid residues that is at least 90% identical to an amino acid sequence selected from the group consisting of: (a) the amino acid sequence as shown in SEQ ID NO:2 from amino acid number 32 (His), to amino acid number 253 (Phe); and (b) the amino acid sequence as shown in SEQ ID NO:2 from amino acid number 1 (Met), to amino acid number 253 (Phe). In one embodiment, the isolated polypeptide disclosed above comprises a sequence of amino acid residues selected from the group consisting of: (a) the amino acid sequence as shown in SEQ ID NO:2 from amino acid number 32 (His), to amino acid number 253 (Phe); and (b) the amino acid sequence as shown in SEQ ID NO:2 from amino acid number 1 (Met), to amino acid number 253 (Phe).

Within another aspect, the present invention provides a method of producing a polypeptide comprising: culturing a cell as disclosed above; and isolating the polypeptide produced by the cell.

Within another aspect, the present invention provides a method of producing an antibody comprising: inoculating an animal with a polypeptide selected from the group consisting of: (a) a polypeptide as disclosed above; (b) a polypeptide comprising the amino acid sequence of SEQ ID NO:2 from amino acid number 42 (Leu), to amino acid number 56 (Ile); (c) a polypeptide comprising the amino acid sequence of SEQ ID NO:2 from amino acid number 108 (Tyr), to amino acid number 122 (Thr); (d) a polypeptide comprising the amino acid sequence of SEQ ID NO:2 from amino acid number 151 (Ile), to amino acid number 165 (Gln); (e) a polypeptide comprising the amino acid sequence of SEQ ID NO:2 from amino acid number 213 (Ile), to amino acid number 227 (Ala); (f) a polypeptide comprising the amino acid sequence of SEQ ID NO:2 from amino acid number 42 (Ile), to amino acid number 227 (Ala); (g) a polypeptide comprising the amino acid sequence of SEQ ID NO:2 from amino acid number 34 (Gln) to amino acid number 39 (Arg); (h) a polypeptide comprising the amino acid sequence of SEQ ID NO:2 from amino acid number 59 (Asn) to amino acid number 64 (Asp); (i) a polypeptide comprising the amino acid sequence of SEQ ID NO:2 from amino acid number 63 (Lys) of SEQ ID NO:2; and (4) amino acid number 116 (Gly); (j) a polypeptide comprising the amino acid sequence of SEQ ID NO:2 from amino acid number 58 (Ala) to amino acid number 121 (Glu); (k) a polypeptide comprising the amino acid sequence of SEQ ID NO:2 from amino acid number 101 (Glu) to amino acid number 107 (Leu); (1) a polypeptide comprising the amino acid sequence of SEQ ID NO:2 from amino acid number 162 (Thr) to amino acid number 169 (Glu); (m) a polypeptide comprising the amino acid sequence of SEQ ID NO:2 from amino acid number 194 (Lys) to amino acid number 200 (Leu); (n) a polypeptide comprising the amino acid sequence of SEQ ID NO:2 from amino acid number 218 (Cys) to amino acid number 225 (Asp); (o) a polypeptide comprising the amino acid sequence of SEQ ID NO:2 from amino acid number Ala (249) to amino acid number 252 (Arg); and wherein the polypeptide elicits an immune response in the animal to produce the antibody; and isolating the antibody from the animal.

Within another aspect, the present invention provides an antibody produced by the method as disclosed above, which binds to a polypeptide as shown in SEQ ID NO:2 from amino acid 32-353. In one embodiment, the antibody disclosed above is a monoclonal antibody. Within another aspect, the present invention provides an antibody that specifically binds to a polypeptide as disclosed above.

Within another aspect, the present invention provides a method of detecting, in a test sample, the presence of an antagonist of zlmda24 protein activity, comprising: culturing a cell that is responsive to a zlmda24-stimulated cellular pathway; and producing a zlmda24 polypeptide by the method as disclosed above; and exposing the zlmda24 polypeptide to the cell, in the presence and absence of a test sample; and comparing levels of response to the zlmda24 polypeptide, in the presence and absence of the test sample, by a biological or biochemical assay; and determining from the comparison, the presence of the antagonist of zlmda24 activity in the test sample.

Within another aspect, the present invention provides a method of detecting, in a test sample, the presence of an agonist of zlmda24 protein activity, comprising: culturing a cell that is responsive to a zlmda24-stimulated cellular pathway; and adding a test sample; and comparing levels of response in the presence and absence of the test sample, by a biological or biochemical assay; and determining from the comparison, the presence of the agonist of zlmda24 activity in the test sample.

Within another aspect, the present invention provides a method for detecting a genetic abnormality in a patient, comprising: obtaining a genetic sample from a patient; producing a first reaction product by incubating the genetic sample with a polynucleotide comprising at least 14 contiguous nucleotides of SEQ ID NO:1 or the complement of SEQ ID NO:1, under conditions wherein said polynucleotide will hybridize to complementary polynucleotide sequence; visualizing the first reaction product; and comparing said first reaction product to a control reaction product from a wild type patient, wherein a difference between said first reaction product and said control reaction product is indicative of a genetic abnormality in the patient.

Within another aspect, the present invention provides a method for detecting testis tissue in a patient sample, comprising: obtaining a tissue or biological sample from a patient; incubating the tissue or biological sample with an antibody as disclosed above under conditions wherein the antibody binds to its complementary polypeptide in the tissue or biological sample; visualizing the antibody bound in the tissue or biological sample; and comparing levels and localization of antibody bound in the tissue or biological sample from the patient to a non-testis control tissue or biological sample, wherein an increase in the level or localization of antibody bound to the patient tissue or biological sample relative to the non-testis control tissue or biological sample is indicative of testis tissue in a patient sample.

Within another aspect, the present invention provides a method for detecting a testicular cancer in a patient, comprising: obtaining a tissue or biological sample from a patient; incubating the tissue or biological sample with an antibody as disclosed above under conditions wherein the antibody binds to its complementary polypeptide in the tissue or biological sample; visualizing the antibody bound in the tissue or biological sample; and comparing levels of antibody bound in the tissue or biological sample from the patient to a normal control tissue or biological sample, wherein an increase in the level of antibody bound to the patient tissue or biological sample relative to the normal control tissue or biological sample is indicative of a testicular cancer in the patient.

Within another aspect, the present invention provides method for detecting testis tissue in a patient sample, comprising: obtaining a tissue or biological sample from a patient; labeling a polynucleotide comprising at least 14 contiguous nucleotides of SEQ ID NO:1 or the complement of SEQ ID NO:1; incubating the tissue or biological sample with under conditions wherein the polynucleotide will hybridize to complementary polynucleotide sequence; visualizing the labeled polynucleotide in the tissue or biological sample; and comparing the level and localization of labeled polynucleotide hybridization in the tissue or biological sample from the patient to a control non-testis tissue or biological sample, wherein an increase in the level or localization of the labeled polynucleotide hybridization to the patient tissue or biological sample relative to the control non-testis tissue or biological sample is indicative of testis tissue in a patient sample.

Within another aspect, the present invention provides a method for detecting a testicular cancer in a patient, comprising: obtaining a tissue or biological sample from a patient; labeling a polynucleotide comprising at least 14 contiguous nucleotides of SEQ ID NO:1 or the complement of SEQ ID NO:1; incubating the tissue or biological sample with under conditions wherein the polynucleotide will hybridize to complementary polynucleotide sequence; visualizing the labeled polynucleotide in the tissue or biological sample; and comparing the level of labeled polynucleotide hybridization in the tissue or biological sample from the patient to a normal control tissue or biological sample, wherein an increase in the labeled polynucleotide hybridization to the patient tissue or biological sample relative to the normal control tissue or biological sample is indicative of a testicular cancer in the patient.

These and other aspects of the invention will become evident upon reference to the following detailed description of the invention.

Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions.

For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of a polynucleotide molecule” denotes a polynucleotide molecule having a complementary base sequence and reverse orientation as compared to a reference sequence. For example, the sequence 5′ ATGCACGGG 3′ is complementary to 5° CCCGTGCAT 3′.

The term “contig” denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of f invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence that encodes a secreted small MW protein (about 27 kD) having a four-helical-bundle structure. Through processes of cloning, and expression studies described herein, a polynucleotide sequence encoding a novel ligand polypeptide has been identified. This polypeptide ligand, designated zlmda24, was isolated from a testis library. Based on RT-PCR analysis, zlmda24 polynucleotides are specifically expressed in testis, and very weakly in salivary gland, and do not appear to be expressed in other tissue types tested. A human zlmda24 polynucleotide sequence is represented in SEQ ID NO:1, and the corresponding polynucleotide sequence is shown in SEQ ID NO:2. An orthologous mouse zlmda24 polynucleotide sequence is represented in SEQ ID NO:3, and the corresponding polynucleotide sequence is shown in SEQ ID NO:4.

Analysis of SEQ ID NO:1 reveals a full-length human polypeptide shown in SEQ ID NO:2. Based on structural similarity of the zlmda24 sequence to known four-helix bundle cytokines, and the presence of a signal sequence, SEQ ID NO:2 encodes a fully functional secreted polypeptide with a helical structure indicative of cytokine-like molecules. Sequence analysis of the human zlmda24 shows a deduced amino acid sequence as represented in SEQ ID NO:2 indicates a 253 amino acid polypeptide containing a 31 amino acid residue secretory signal sequence (amino acid residues 1 (Met) to 31 (Leu) of SEQ ID NO:2), and a mature polypeptide of 222 amino acids (amino acid residues 32 (His) to 253 (Phe) of SEQ ID NO:2). A mouse ortholog was also isolated and is shown in SEQ ID NO:3 and SEQ ID NO:4 and is discussed in further detail below.

In general, cytokines are predicted to have a four-alpha helix structure, with the 1^{st} and 4^{th} helices being most important in ligand-receptor interactions. The 1^{st} and 4^{th} helices are more highly conserved among members of the family. Referring to the human zlmda24 amino acid sequence shown in SEQ ID NO:2, comparison of human zlmda24, and known cytokines, such as human IL-10, human zcyto10 (WO US98/25228), and human MDA7 (Genbank Accession No. Q13007), IL-15, IL-2, IL-4, IL-6 and GM-CSF amino acid sequences suggests that human zlmda24 helix A is defined by amino acid residues 42 (Leu) to 56 (Ile) of SEQ ID NO:2; helix B by amino acid residues 108 (Tyr) to 122 (Thr) of SEQ ID NO:2; helix C by amino acid residues 151 (Ile) to 165 (Gln) of SEQ ID NO:2; and helix D by amino acid residues 213 (Ile) to 227 (Ala) of SEQ ID NO:2. Structural analysis suggests that the A/B loop is long, the B/C loop is short and the C/D loop is long. This loop structure results in an up-up-down-down helical organization.

The corresponding polynucleotides encoding the human zlmda24 polypeptide regions, domains, motifs, residues and sequences described herein are as shown in SEQ ID NO:1. Moreover, the corresponding zlmda24 polypeptide regions, domains, motifs, residues and sequences described herein are also as shown in SEQ ID NO:2.

Four-helical bundle cytokines are also grouped by the length of their component helices. “Long-helix” form cytokines generally consist of between 24-30 residue helices and include IL-6, ciliary neutrotrophic factor (CNTF), leukemia inhibitory factor (LIF) and human growth hormone (hGH). “Short-helix” form cytokines generally consist of between 18-21 residue helices and include IL-2, IL-4 and GM-CSF. Zlmda24 is believed to be a new member of the short-helix form cytokine group. Studies using CNTF and IL-6 demonstrated that a CNTF helix can be exchanged for the equivalent helix in IL-6, conferring CTNF-binding properties to the chimera. Thus, it appears that functional domains of four-helical cytokines determined on the basis of structural homology, irrespective of sequence identity, and can maintain functional integrity in a chimera (Kallen et al., J. Biol. Chem. 274:11859-11867, 1999). Using similar methods, putative regions conferring receptor binding specificity in zlmda24 comprise the regions of amino acid residues of SEQ ID NO:2 that include: residues 42-56 (helix A), 108-122 (Helix B), 151-165 (Helix C), and residues 213-227 (helix D); and more preferably residues 42-56 (helix A) and residues 213-227 (helix D). These regions will be useful for preparing chimeric molecules, particularly with other short-helix form cytokines to determine and modulate receptor binding specificity.

The present invention provides polynucleotide molecules, including DNA and RNA molecules that encode the zlmda24 polypeptides disclosed herein. Those skilled in the art will readily recognize that, in view of the degeneracy of the genetic code, considerable sequence variation is possible among these polynucleotide molecules. SEQ ID NO:5 is a degenerate DNA sequence that encompasses all DNAs that encode the human zlmda24 polypeptide of SEQ ID NO:2. SEQ ID NO:6 is a degenerate DNA sequence that encompasses all DNAs that encode the mouse zlmda24 polypeptide of SEQ ID NO:4. Those skilled in the art will recognize that the degenerate sequence of SEQ ID NO:5 and SEQ ID NO:6 also provides all RNA sequences encoding SEQ ID NO:2 and SEQ ID NO:4 by substituting U for T. Thus, zlmda24 polypeptide-encoding polynucleotides comprising nucleotide 1 or 94 to nucleotide 759 of SEQ ID NO:5, and polynucleotides comprising nucleotide 1 or 82 to nucleotide 759 of SEQ ID NO:6, and their RNA equivalents are contemplated by the present invention. Table 1 sets forth the one-letter codes used within SEQ ID NO:5 to denote degenerate nucleotide positions. “Resolutions” are the nucleotides denoted by a code letter. “Complement” indicates the code for the complementary nucleotide(s). For example, the code Y denotes either C or T, and its complement R denotes A or G, with A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:5, encompassing all possible codons for a given amino acid, are set forth in Table 2.

TABLE 2

| | One | | |
|---|---|---|---|
| Amino Letter | | Degenerate | |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | B | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2 or SEQ ID NO:4. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed in SEQ ID NO:5 and SEQ ID NO:6 serve as a template for optimizing expression of polynucleotides in various cell types and species commonly used in the art and disclosed herein. Sequences containing preferential codons can be tested and optimized for expression in various species, and tested for functionality as disclosed herein.

As previously noted, the isolated polynucleotides of the present invention include DNA and RNA. Methods for preparing DNA and RNA are well known in the art. In general, RNA is isolated from a tissue or cell that produces large amounts of zlmda24 RNA, or is a tissue or cell that specifically expresses zlmda24 such as testis tissue or cells, including whole testis tissue extracts or testicular cells, such as Sertoli cells, Leydig cells, spermatogonia, or epididymis, cells from vas deferens, and the like, although DNA can also be prepared using RNA from other tissues or isolated as genomic DNA. Such tissues and cells are identified by Northern blotting (Thomas, Proc. Natl. Acad. Sci. USA 77:5201, 1980), reverse transcriptase PCR (RT-PCR) or by screening conditioned medium from various cell types for activity on target cells or tissue. Once the activity or RNA producing cell or tissue is identified, total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding zlmda24 polypeptides are then identified and isolated by, for example, hybridization or PCR.

A full-length clone encoding zlmda24 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zlmda24 fragments, or other specific binding partners.

Zlmda24 polynucleotide sequences disclosed herein can also be used as probes or primers to clone 5′ non-coding regions of a zlmda24 gene. In view of the tissue-specific expression observed for zlmda24 by Northern blotting and RT PCR (See, Examples 2 and 3), this gene region is expected to provide for testis-specific expression. Promoter elements from a zlmda24 gene could thus be used to direct the tissue-specific expression of heterologous genes in, for example, transgenic animals or patients treated with gene therapy. Cloning of 5′ flanking sequences also facilitates production of zlmda24 proteins by “gene activation” as disclosed in U.S. Pat. No. 5,641,670. Briefly, expression of an endogenous zlmda24 gene in a cell is altered by introducing into the zlmda24 locus a DNA construct comprising at least a targeting sequence, a regulatory sequence, an exon, and an unpaired splice donor site. The targeting sequence is a zlmda24 5′ non-coding sequence that permits homologous recombination of the construct with the endogenous zlmda24 locus, whereby the sequences within the construct become operably linked with the endogenous zlmda24 coding sequence. In this way, an endogenous zlmda24 promoter can be replaced or supplemented with other regulatory sequences to provide enhanced, tissue-specific, or otherwise regulated expression.

The present invention further provides counterpart polypeptides and polynucleotides from other species (orthologs). These species include, but are not limited to mammalian, avian, amphibian, reptile, fish, insect and other vertebrate and invertebrate species. Of particular interest are zlmda24 polypeptides from other mammalian species, including murine, porcine, ovine, bovine, canine, feline, equine, and other primate polypeptides. Orthologs of human zlmda24 can be cloned using information and compositions provided by the present invention in combination with conventional cloning techniques. For example, a cDNA can be cloned using mRNA obtained from a tissue or cell type that expresses zlmda24 such as testis, as disclosed herein. Suitable sources of mRNA can be identified by probing Northern blots with probes designed from the sequences disclosed herein. A library is then prepared from mRNA of a positive tissue or cell line. A zlmda24-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human zlmda24 sequence disclosed herein. Within an additional method, the cDNA library can be used to transform or transfect host cells, and expression of the cDNA of interest can be detected with an antibody to zlmda24 polypeptide, binding studies or activity assays. Similar techniques can also be applied to the isolation of genomic clones. Example 1 shows that a zlmda24 ortholog is expressed in mouse testis.

The polynucleotide sequence for the mouse ortholog of zlmda24 has been identified in mouse testis and is shown in SEQ ID NO:3 and the corresponding amino acid sequence shown in SEQ ID NO:4. There is a 70% identity between the mouse and human zlmda24 sequences over a 253 amino acid region that corresponds to residues 1 to 253 in SEQ ID NO:2 and residues 1 to 253 of SEQ ID NO:4. Sequence analysis of the deduced amino acid sequence of the mouse zlmda24 polypeptide as represented in SEQ ID NO:4 indicates a 253 amino acid polypeptide containing a 27 amino acid residue secretory signal sequence (amino acid residues 1 (Met) to 27 (Arg) of SEQ ID NO:4), and a mature polypeptide of 226 amino acids (amino acid residues 28 (Thr) to 253 (Phe) of SEQ ID NO:2). Moreover, similar to the human sequence, the mouse has a four-helix bundle structure with helix A defined by amino acid residues 58 (Gly) to 72 (Leu) of SEQ ID NO:4; helix B by amino acid residues 108 (Phe) to 122 (Thr) of SEQ ID NO:4; helix C by amino acid residues 144 (Met) to 158 (Leu) of SEQ ID NO:4; and helix D by amino acid residues 213 (Ile) to 227 (Ala) of SEQ ID NO:4.

The corresponding polynucleotides encoding the mouse zlmda24 polypeptide regions, domains, motifs, residues and sequences described herein are as shown in SEQ ID NO:3. Moreover, the corresponding zlmda24 polypeptide regions, domains, motifs, residues and sequences described herein are also as shown in SEQ ID NO:4.

Those skilled in the art will recognize that the sequence disclosed in SEQ ID NO:1 represents a single allele of human zlmda24 and that allelic variation and alternative splicing are expected to occur. Allelic variants of this sequence can be cloned by probing cDNA or genomic libraries from different individuals according to standard procedures. Allelic variants of the DNA sequence shown in SEQ ID NO:1, including those containing silent mutations and those in which mutations result in amino acid sequence changes, are within the scope of the present invention, as are proteins which are allelic. variants of SEQ ID NO:2. cDNAs generated from alternatively spliced mRNAs, which retain the properties of the zlmda24 polypeptide, are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

Moreover, the genomic structure of zlmda24 is readily determined by one of skill in the art by comparing the cDNA sequence of SEQ ID NO:1 and the translated amino acid of SEQ ID NO:2 with the genomic DNA in which the gene is contained (Genbank Accession No.'s AC073457, and AC002127). For example, such analysis can be readily done using FASTA as described herein. As such, the intron and exon junctions in this region of genomic DNA can be determined for the zlmda24 gene. Thus, the present invention includes the zlmda24 gene as located in human genomic DNA. Moreover, the zlmda24 gene is located at the 7q21 region of human chromosome 7 (Example 3).

Within preferred embodiments of the invention, isolated zlmda24-encoding nucleic acid molecules can hybridize under stringent conditions to nucleic acid molecules having the nucleotide sequence of SEQ ID NO:1, to nucleic acid molecules having the nucleotide sequence of nucleotides 205-962, or 298-962 of SEQ ID NO:1, or to nucleic acid molecules having a nucleotide sequence complementary to 205-962, or 298-962 of SEQ ID NO:1. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe.

A pair of nucleic acid molecules, such as DNA-DNA, RNA-RNA and DNA-RNA, can hybridize if the nucleotide sequences have some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the Tₘ of the hybrid and a hybridization buffer having up to 1 M Na⁺. Higher degrees of stringency at lower temperatures can be achieved with the addition of formamide which reduces the Tₘ of the hybrid about 1° C. for each 1% formamide in the buffer solution. Generally, such stringent conditions include temperatures of 20-70° C. and a hybridization buffer containing up to 6×SSC and 0-50% formamide. A higher degree of stringency can be achieved at temperatures of from 40-70° C. with a hybridization buffer having up to 4×SSC and from 0-50% formamide. Highly stringent conditions typically encompass temperatures of 42-70° C. with a hybridization buffer having up to 1×SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes.

The above conditions are meant to serve as a guide, and it is well within the abilities of one skilled in the art to adapt these conditions for use with a particular polynucleotide hybrid. The Tₘ for a specific target sequence is the temperature (under defined conditions) at which 50% of the target sequence will hybridize to a perfectly matched probe sequence. Those conditions which influence the Tₘ include, the size and base pair content of the polynucleotide probe, the ionic strength of the hybridization solution, and the presence of destabilizing agents in the hybridization solution. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tₘ or 5-10° C. below the calculated Tₘ. This allows for the maximum rate of hybridization for DNA-DNA and DNA-RNA hybrids.

The length of the polynucleotide sequence influences the rate and stability of hybrid formation. Smaller probe sequences, <50 base pairs, reach equilibrium with complementary sequences rapidly, but may form less stable hybrids. Incubation times of anywhere from minutes to hours can be used to achieve hybrid formation. Longer probe sequences come to equilibrium more slowly, but form more stable complexes, even at lower temperatures. In such cases, incubations are allowed to proceed overnight or longer. Generally, incubations are carried out for a period equal to three times the calculated Cot time. Cot time, the time it takes for the polynucleotide sequences to reassociate, can be calculated for a particular sequence by methods known in the art.

The base pair composition of a polynucleotide sequence will affect the thermal stability of its hybrid complex, thereby influencing the choice of hybridization temperature and the ionic strength of the hybridization buffer. A-T pairs are less stable than G-C pairs in aqueous solutions containing sodium chloride. Therefore, the higher the G-C content, the more stable the hybrid. Even distribution of G and C residues within the sequence also contributes positively to hybrid stability. In addition, the base pair composition can be manipulated to alter the Tₘ of a given sequence. For example, 5-methyldeoxycytidine can be substituted for deoxycytidine and 5-bromodeoxuridine can be substituted for thymidine to increase the Tₘ, whereas 7-deazzo-2′-deoxyguanosine can be substituted for guanosine to reduce dependence on Tₘ.

The ionic concentration of the hybridization buffer also affects the stability of the hybrid. Hybridization buffers generally contain blocking agents such as Denhardt's solution (Sigma Chemical Co., St. Louis, Mo.), denatured salmon sperm DNA, tRNA, milk powders (BLOTTO), heparin or SDS, and a Na⁺ source, such as SSC (1×SSC: 0.15 M sodium chloride, 15 mM sodium citrate) or SSPE (1×SSPE: 1.8 M NaCl, 10 mM NaH₂PO₄, 1 mM EDTA, pH 7.7). By decreasing the ionic concentration of the buffer, the stability of the hybrid is increased. Typically, hybridization buffers contain from between 10 mM-1 M Na⁺. The addition of destabilizing or denaturing agents such as formamide, tetralkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide also acts to reduce non-specific background when using RNA probes.

As an illustration, a nucleic acid molecule encoding a variant zlmda24 polypeptide can be hybridized with a nucleic acid molecule having the nucleotide sequence of SEQ ID NO:1 (or its complement) at 42° C. overnight in a solution comprising 50% formamide, 5×SSC (1×SSC: 0.15 M sodium chloride and 15 mM sodium citrate), 50 mM sodium phosphate (pH 7.6), 5×Denhardt's solution (100×Denhardt's solution: 2% (w/v) Ficoll 400, 2% (w/v) polyvinylpyrrolidone, and 2% (w/v) bovine serum albumin), 10% dextran sulfate, and 20 μg/ml denatured, sheared salmon sperm DNA. One of skill in the art can devise variations of these hybridization conditions. For example, the hybridization mixture can be incubated at a higher temperature, such as about 65° C., in a solution that does not contain formamide. Moreover, premixed hybridization solutions are available (e.g., EXPRESSHYB Hybridization Solution from CLONTECH Laboratories, Inc.), and hybridization can be performed according to the manufacturer's instructions.

Following hybridization, the nucleic acid molecules can be washed to remove non-hybridized nucleic acid molecules under stringent conditions, or under highly stringent conditions. Typical stringent washing conditions include washing in a solution of 0.5×-2×SSC with 0.1% sodium dodecyl sulfate (SDS) at 55-65° C. That is, nucleic acid molecules encoding a variant zlmda24 polypeptide hybridize with a nucleic acid molecule having the nucleotide sequence of SEQ ID NO:1 (or its complement) under stringent washing conditions, in which the wash stringency is equivalent to 0.5×-2×SSC with 0.1% SDS at 55-65° C., including 0.5×SSC with 0.1% SDS at 55° C., or 2×SSC with 0.1% SDS at 65° C. One of skill in the art can readily devise equivalent conditions, for example, by substituting SSPE for SSC in the wash solution.

Typical highly stringent washing conditions include washing in a solution of 0.1×-0.2×SSC with 0.1% sodium dodecyl sulfate (SDS) at 50-65° C. In other words, nucleic acid molecules encoding a variant zlmda24 polypeptide hybridize with a nucleic acid molecule having the nucleotide sequence of SEQ ID NO:1 (or its complement) under highly stringent washing conditions, in which the wash stringency is equivalent to 0.1×-0.2×SSC with 0.1% SDS at 50-65° C., including 0.1×SSC with 0.1% SDS at 50° C., or 0.2×SSC with 0.1% SDS at 65° C.

The present invention also provides isolated zlmda24 polypeptides that have a substantially similar sequence identity to the polypeptides of SEQ ID NO:2, or their orthologs. The term “substantially similar sequence identity” is used herein to denote polypeptides comprising at least 70%, at least 80%, at least 90%, at least 95%, or greater than 95% sequence identity to the sequences shown in SEQ ID NO:2, or their orthologs. The present invention also includes polypeptides that comprise an amino acid sequence having at least 70%, at least 80%, at least 90%, at least 95% or greater than 95% sequence identity to the sequence of amino acid residues 1-253, or 32-253 of SEQ ID NO:2; or amino acid residues 1 to 253, or 28-253 of SEQ ID NO:4. The present invention further includes nucleic acid molecules that encode such polypeptides. Methods for determining percent identity are described below.

The present invention also contemplates variant zlmda24 nucleic acid molecules that can be identified using two criteria: a determination of the similarity between the encoded polypeptide with the amino acid sequence of SEQ ID NO:2, and/or a hybridization assay, as described above. Such zlmda24 variants include nucleic acid molecules: (1) that hybridize with a nucleic acid molecule having the nucleotide sequence of SEQ ID NO:1 (or its complement) under stringent washing conditions, in which the wash stringency is equivalent to 0.5×-2×SSC with 0.1% SDS at 55-65° C.; or (2) that encode a polypeptide having at least 70%, at least 80%, at least 90%, at least 95% or greater than 95% sequence identity to the amino acid sequence of SEQ ID NO:2. Alternatively, zlmda24 variants can be characterized as nucleic acid molecules: (1) that hybridize with a nucleic acid molecule having the nucleotide sequence of SEQ ID NO:1 (or its complement) under highly stringent washing conditions, in which the wash stringency is equivalent to 0.1×-0.2×SSC with 0.1% SDS at 50-65° C.; and (2) that encode a polypeptide having at least 70%, at least 80%, at least 90%, at least 95% or greater than 95% sequence identity to the amino acid sequence of SEQ ID NO:2.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 3 (amino acids are indicated by the standard one-letter codes).

$$\frac{ Total number of identical matches }{\begin{matrix} [ length of the longer sequence plus the \\ number of gaps introduced into the longer \\ sequence in order to align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zlmda24. The FASTA algorithm is described by Pearson and Lipman, Proc. Nat'l Acad. Sci. USA 85:2444 (1988), and by Pearson, Meth. Enzymol. 183:63 (1990).

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other FASTA parameters set as default.

Variant zlmda24 polypeptides or polypeptides with substantially similar sequence identity are characterized as having one or more amino acid substitutions, deletions or additions. These changes are preferably of a minor nature, that is conservative amino acid substitutions (see Table 4) and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from about 200 to about 280 amino acid residues that comprise a sequence that is at least 70%, preferably at least 90%, and more preferably 95% or more identical to the corresponding region of SEQ ID NO:2. Polypeptides comprising affinity tags can further comprise a proteolytic cleavage site between the zlmda24 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Determination of amino acid residues that comprise regions or domains that are critical to maintaining structural integrity can be determined. Within these regions one can determine specific residues that will be more or less tolerant of change and maintain the overall tertiary structure of the molecule. Methods for analyzing sequence structure include, but are not limited to alignment of multiple sequences with high amino acid or nucleotide identity, secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in zlmda24 polypeptides so as to minimize disruption of higher order structure essential to biological activity. For example, when the zlmda24 polypeptide comprises one or more helices, changes in amino acid residues will be made so as not to disrupt the helix geometry and other components of the molecule where changes in conformation abate some critical function, for example, an active site, or binding of the molecule to its binding partners. As such, preferably, zlmda24 helices A and D would not be disrupted. The effects of amino acid sequence changes can be predicted by, for example, computer modeling as disclosed above or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in variant and standard molecules can be made. Mass spectrometry and chemical modification using reduction and alkylation provide methods for determining cysteine residues that are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same cysteine pattern as the standard molecule folding would be affected. Another well known and accepted method for measuring folding is circular dichroism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structurally similarities between proteins and polypeptides (Schaanan et al., Science 257:961-964, 1992).

A Hopp/Woods hydrophilicity profile of the zlmda24 protein sequence as shown in SEQ ID NO:2 or SEQ ID NO:4 can be generated (Hopp et al., Proc. Natl. Acad. Sci.78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. For example, in zlmda24, hydrophilic regions include: (1) amino acid number 34 (Gln) to amino acid number 39 (Arg) of SEQ ID NO:2; (2) amino acid number 59 (Asn) to amino acid number 64 (Asp) of SEQ ID NO:2; (3) amino acid number 58 (Ala) to amino acid number 63 (Lys) of SEQ ID NO:2; and (4) amino acid number 116 (Gly) to amino acid number 121 (Glu) of SEQ ID NO:2; and (1) amino acid number 34 (Gin) to amino acid number 39 (Arg) of SEQ ID NO:4; (2) amino acid number 59 (Asn) to amino acid number 64 (Asp) of SEQ ID NO:4; (3) amino acid number 159 (Asp) to amino acid number 164 (Asp) of SEQ ID NO:4; and (4) amino acid number 118 (Glu) to amino acid number 123 (Glu) of SEQ ID NO:4.

Those skilled in the art will recognize that hydrophilicity or hydrophobicity will be taken into account when designing modifications in the amino acid sequence of a zlmda24 polypeptide, so as not to disrupt the overall structural and biological profile. Of particular interest for replacement are hydrophobic residues selected from the group consisting of Val, Leu and Ile or the group consisting of Met, Gly, Ser, Ala, Tyr and Trp. For example, residues tolerant of substitution could include such residues as shown in SEQ ID NO:2 or SEQ ID NO:4. Cysteine residues of SEQ ID NO:2, will be relatively intolerant of substitution.

The identities of essential amino acids can also be inferred from analysis of sequence similarity between human and mouse zlmda24, and with reference to known four helix bundle cytokines such as IL-10, zmda7, IL-2, IL-4, IL-6, and the like. Using methods such as “FASTA” analysis described previously, regions of high similarity are identified within a family of proteins and used to analyze amino acid sequence for conserved regions. An alternative approach to identifying a variant zlmda24 polynucleotide on the basis of structure is to determine whether a nucleic acid molecule encoding a potential variant zlmda24 gene can hybridize to a nucleic acid molecule having the nucleotide sequence of SEQ ID NO:1, as discussed above. Similarly, these approaches described herein can be used to identify a molecule encoding a variant mouse zlmda24.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699 (1996).

The present invention also includes functional fragments of zlmda24 polypeptides and nucleic acid molecules encoding such functional fragments. A “functional” zlmda24 or fragment thereof as defined herein is characterized by its proliferative or differentiating activity, by its ability to induce or inhibit specialized cell functions, or by its ability to bind specifically to an anti-zlmda24 antibody, cell, or zlmda24 receptor (either soluble or immobilized). As previously described herein, human zlmda24 is characterized by a four-helical-bundle structure comprising helix A (amino acid residues 42-56), helix B (amino acid residues 108-122), helix C (amino acid residues 151-165) and helix D (amino acid residues 213-227), as shown in SEQ ID NO:2. The helices A-D as shown in SEQ ID NO:4 for the mouse zlmda24 sequence are described herein. Thus, the present invention further provides fusion proteins encompassing: (a) polypeptide molecules comprising one or more of the helices described above; and (b) functional fragments comprising one or more of these helices. The other polypeptide portion of the fusion protein may be contributed by another four-helical-bundle cytokine, such as IL-10, zcytolo, MDA7, IL-15, IL-2, IL-4 and GM-CSF, or by a non-native and/or an unrelated secretory signal peptide that facilitates secretion of the fusion protein.

Routine deletion analyses of nucleic acid molecules can be performed to obtain functional fragments of a nucleic acid molecule that encodes a zlmda24 polypeptide. As an illustration, DNA molecules having the nucleotide sequence of SEQ ID NO:1 or SEQ ID NO:3 or fragments thereof, can be digested with Bal31 nuclease to obtain a series of nested deletions. These DNA fragments are then inserted into expression vectors in proper reading frame, and the expressed polypeptides are isolated and tested for zlmda24 activity, or for the ability to bind anti-zlmda24 antibodies or zlmda24 receptor. One alternative to exonuclease digestion is to use oligonucleotide-directed mutagenesis to introduce deletions or stop codons to specify production of a desired zlmda24 fragment. Alternatively, particular fragments of a zlmda24 gene can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993); Content et al., “Expression and preliminary deletion analysis of the 42 kDa 2-SA synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation 1, Boynton et al., (eds.) pages 169-199 (Academic Press 1985); Coumailleau et al., J. Biol. Chem. 270:29270 (1995); Fukunaga et al., J. Biol. Chem. 270:25291 (1995); Yamaguchi et al., Biochem. Pharmacol. 50:1295 (1995); and Meisel et al., Plant Molec. Biol. 30:1 (1996).

Multiple amino acid substitutions can be made and tested using known methods of mutagenesis and screening, such as those disclosed by Reidhaar-Olson and Sauer (Science 241:53 (1988)) or Bowie and Sauer (Proc. Nat'l Acad. Sci. USA 86:2152 (1989)). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832 (1991), Ladner et al., U.S. Pat. No. 5,223,409, Huse, international publication No. WO 92/06204), and region-directed mutagenesis (Derbyshire et al., Gene 46:145 (1986), and Ner et al., DNA 7:127, (1988)).

Variants of the disclosed zlmda24 nucleotide and polypeptide sequences can also be generated through DNA shuffling as disclosed by Stemmer, Nature 370:389 (1994), Stemmer, Proc. Natl Acad. Sci. USA 91:10747 (1994), and international publication No. WO 97/20078. Briefly, variant DNA molecules are generated by in vitro homologous recombination by random fragmentation of a parent DNA followed by reassembly using PCR, resulting in randomly introduced point mutations. This technique can be modified by using a family of parent DNA molecules, such as allelic variants or DNA molecules from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode biologically active polypeptides, or polypeptides that bind with anti-zlmda24 antibodies or soluble zlmda24 receptor, can be recovered from the host cells and rapidly sequenced using modem equipment. These methods allow the rapid determination of the importance of individual amino acid residues in a polypeptide of interest, and can be applied to polypeptides of unknown structure.

In addition, the proteins of the present invention (or polypeptide fragments thereof) can be joined to other bioactive molecules, particularly other cytokines, to provide multi-functional molecules. For example, one or more helices from zlmda24 can be joined to other cytokines to enhance their biological properties or efficiency of production.

The present invention thus provides a series of novel, hybrid molecules in which a segment comprising one or more of the helices of zlmda24 is fused to another polypeptide. Fusion is preferably done by splicing at the DNA level to allow expression of chimeric molecules in recombinant production systems. The resultant molecules are then assayed for such properties as improved solubility, improved stability, prolonged clearance half-life, improved expression and secretion levels, and pharmacodynamics. Such hybrid molecules may further comprise additional amino acid residues (e.g. a polypeptide linker) between the component proteins or polypeptides.

Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722 (1991), Ellman et al., Methods Enzymol. 202:301 (1991), Chung et al., Science 259:806 (1993), and Chung et al., Proc. Nat'l Acad. Sci. USA 90:10145 (1993).

In a second method, translation is carried out in Xenopus oocytes by microinjection of mutated mRNA and chemically aminoacylated suppressor tRNAs (Turcatti et al., J. Biol. Chem. 271:19991 (1996)). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470 (1994). Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395 (1993)).

A limited number of non-conservative amino acids, amino acids that are not encoded by the genetic code, non-naturally occurring amino acids, and unnatural amino acids may be substituted for zlmda24 amino acid residues.

The present invention also provides polypeptide fragments or peptides comprising an epitope-bearing portion of a zlmda24 polypeptide described herein.

Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983)).

In contrast, polypeptide fragments or peptides may comprise an “antigenic epitope,” which is a region of a protein molecule to which an antibody can specifically bind. Certain epitopes consist of a linear or contiguous stretch of amino acids, and the antigenicity of such an epitope is not disrupted by denaturing agents. It is known in the art that relatively short synthetic peptides that can mimic epitopes of a protein can be used to stimulate the production of antibodies against the protein (see, for example, Sutcliffe et al., Science 219:660 (1983)). Accordingly, antigenic epitope-bearing peptides and polypeptides of the present invention are useful to raise antibodies that bind with the polypeptides described herein. Hopp/Woods hydrophilicity profiles can be used to determine regions that have the most antigenic potential (Hopp et al., 1981, ibid. and Hopp, 1986, ibid.). In zlmda24 these regions include: (1) amino acid number 34 (Gln) to amino acid number 39 (Arg) of SEQ ID NO:2; (2) amino acid number 59 (Asn) to amino acid number 64 (Asp) of SEQ ID NO:2; (3) amino acid number 58 (Ala) to amino acid number 63 (Lys) of SEQ ID NO:2; and (4) amino acid number 116 (Gly) to amino acid number 121 (Glu) of SEQ I) NO:2; and (1) amino acid number 34 (Gln) to amino acid number 39 (Arg) of SEQ ID NO:4; (2) amino acid number 59 (Asn) to amino acid number 64 (Asp) of SEQ ID NO:4; (3) amino acid number 159 (Asp) to amino acid number 164 (Asp) of SEQ ID NO:4; and (4) amino acid number 118 (Glu) to amino acid number 123 (Glu) of SEQ ID NO:4. Moreover, zlmda24 antigenic epitope-bearing polypeptides as predicted by a Jameson-Wolf plot, e.g., using DNASTAR Protean program (DNASTAR, Inc., Madison, Wis.) are suitable, including polypeptides comprising: (1) amino acid number 7 (Asn) to amino acid number 16 (Pro) of SEQ ID NO:2; (2) amino acid number 60 (Val) to amino acid number 67 (Asp) of SEQ ID NO:2; (3) amino acid number 101 (Glu) to amino acid number 107 (Leu) of SEQ ID NO:2; (4) amino acid number 162 (Thr) to amino acid number 169 (Glu) of SEQ ID NO:2; (5) amino acid number 194 (Lys) to amino acid number 200 (Leu) of SEQ ID NO:2; (6) amino acid number 218 (Cys) to amino acid number 225 (Asp) of SEQ ID NO:2; (7) amino acid number Ala (249) to amino acid number 252 (Arg) of SEQ ID NO:2; and (1) amino acid number 6 (His) to amino acid number 14 (Gln) of SEQ ID NO:4; (2) amino acid number 59 (Asn) to amino acid number 67 (Glu) of SEQ ID NO:4; (3) amino acid number 101 (Glu) to amino acid number 106 (Thr) of SEQ ID NO:4; (4) amino acid number 159 (Asp) to amino acid number 166 (Ser) of SEQ ID NO:4; (5) amino acid number 158 (Leu) to amino acid number 167 (Ile) of SEQ ID NO:4; (6) amino acid number 193 (Ala) to amino acid number 199 (Asn) of SEQ ID NO:4; (7) amino acid number Ala (249) to amino acid number 252 (Arg) of SEQ ID NO:4.

Antigenic epitope-bearing peptides and polypeptides preferably contain at least four to ten amino acids, at least ten to fifteen amino acids, or about 15 to about 30 amino acids of SEQ ID NO:2. Such epitope-bearing peptides and polypeptides can be produced by fragmenting a zlmda24 polypeptide, or by chemical peptide synthesis, as described herein. Moreover, epitopes can be selected by phage display of random peptide libraries (see, for example, Lane and Stephen, Curr. Opin. Immunol. 5:268 (1993); and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992); Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

Regardless of the particular nucleotide sequence of a variant zlmda24 polynucleotide, the polynucleotide encodes a polypeptide that is characterized by its proliferative or differentiating activity, its ability to induce or inhibit specialized cell functions, or by the ability to bind specifically to an anti-zlmda24 antibody or zlmda24 receptor. More specifically, variant zlmda24 polynucleotides will encode polypeptides which exhibit at least 50% and preferably, greater than 70%, 80% or 90%, of the activity of the polypeptide as shown in SEQ ID NO:2.

For any zlmda24 polypeptide, including variants and fusion proteins, one of ordinary skill in the art can readily generate a fully degenerate polynucleotide sequence encoding that variant using the information set forth in Tables 1 and 2 above.

The present invention further provides a variety of other polypeptide fusions (and related multimeric proteins comprising one or more polypeptide fusions). For example, a zlmda24 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin- zlmda24 polypeptide fusions can be expressed in genetically engineered cells (to produce a variety of multimeric zlmda24 analogs). Auxiliary domains can be fused to zlmda24 polypeptides to target them to specific cells, tissues, or macromolecules. For example, a zlmda24 polypeptide or protein could be targeted to a predetermined cell type by fusing a zlmda24 polypeptide to a ligand that specifically binds to a receptor on the surface of that target cell. In this way, polypeptides and proteins can be targeted for therapeutic or diagnostic purposes. A zlmda24 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

Using the methods discussed herein, one of ordinary skill in the art can identify and/or prepare a variety of polypeptides that have substantially similar sequence identity to amino acid residues 1-253 or 32-253 of SEQ ID NO:2, 1-253 or 28-253 of SEQ ID NO:4, or functional fragments and fusions thereof, wherein such polypeptides or fragments or fusions retain the properties of the wild-type protein such as the ability to stimulate proliferation or signal transduction activity, differentiation, induce specialized cell function or bind the zlmda24 receptor or zlmda24 antibodies.

The zlmda24 polypeptides of the present invention, including full-length polypeptides, functional fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a zlmda24 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zlmda24 polypeptide into the secretory pathway of a host cell, a secretory signal sequence (also known as a leader sequence, prepro sequence or pre sequence) is provided in the expression vector. The secretory signal sequence may be that of zlmda24, or may be derived from another secreted protein (e.g., t-PA) or synthesized de novo. The secretory signal sequence is operably linked to the zlmda24 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

Alternatively, the secretory signal sequence contained in the polypeptides of the present invention is used to direct other polypeptides into the secretory pathway. The present invention provides for such fusion polypeptides. A signal fusion polypeptide can be made wherein a secretory signal sequence comprising amino acid residues 1-31 of SEQ ID NO:2, or amino acid residues 1-27 of SEQ ID NO:4 is be operably linked to a DNA sequence encoding another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein. Such fusions may be used in vivo or in vitro to direct peptides through the secretory pathway.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK 30 (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g.

CHO-K1; ATCC No. CCL 61) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Banalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. The second method of making recombinant baculovirus utilizes a transposon-based system described by Luckow (Luckow, V. A., et al., J Virol 67:4566-79, 1993). This system is sold in the Bac-to-Bac kit (Life Technologies, Rockville, Md.). This system utilizes a transfer vector, pFastBac 1™ (Life Technologies) containing a Tn7 transposon to move the DNA encoding the zlmda24 polypeptide into a baculovirus genome maintained in E. coli as a large plasmid called a “bacmid.” The pFastBac1™ transfer vector utilizes the AcNPV polyhedrin promoter to drive the expression of the gene of interest, in this case zlmda24. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol. Chem. 270:1543-9, 1995. In such transfer vector constructs, a short or long version of the basic protein promoter can be used. Moreover, transfer vectors can be constructed which replace the native zlmda24 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native zlmda24 secretory signal sequence. In addition, transfer vectors can include an in-frame fusion with DNA encoding an epitope tag at the C- or N-terminus of the expressed zlmda24 polypeptide, for example, a Glu-Glu epitope tag (Grussenmeyer, T. et al., Proc. Natl. Acad. Sci. 82:7952-4, 1985). Using techniques known in the art, a transfer vector containing zlmda24 is transformed into E. Coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zlmda24 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the zlmda24 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zlmda24 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

It is preferred to purify the polypeptides of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant zlmda24 polypeptides (or chimeric zlmda24 polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their physical properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39) and use of the soluble zlmda24 receptor. Within additional embodiments of the invention, a fusion of the polypeptide of interest and an affinity tag (e.g., maltose-binding protein, Glu-Glu tag, an immunoglobulin domain) may be constructed to facilitate purification.

Moreover, using methods described in the art, polypeptide fusions, or hybrid zlmda24 proteins, are constructed using regions or domains of the inventive zlmda24 in combination with those of other human cytokine family proteins (e.g. interleukins or GM-CSF), or heterologous proteins (Sambrook et al., ibid., Altschul et al., ibid., Picard, Cur. Opin. Biology, 5:511-5, 1994, and references therein). These methods allow the determination of the biological importance of larger domains or regions in a polypeptide of interest. Such hybrids may alter reaction kinetics, binding, alter cell proliferative activity, constrict or expand the substrate specificity, or alter tissue and cellular localization of a polypeptide, and can be applied to polypeptides of unknown structure.

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. For example, part or all of a helix conferring a biological function may be swapped between zlmda24 of the present invention with the functionally equivalent helices from another family member, such as IL-10, zcyto10, MDA7, IL-15, IL-2, IL-4 and GM-CSF. Such components include, but are not limited to, the secretory signal sequence, helices A, B, C, D and four-helical-bundle cytokines. Such fusion proteins would be expected to have a biological functional profile that is the same or similar to polypeptides of the present invention or other known four-helical-bundle cytokine family proteins, depending on the fusion constructed. Moreover, such fusion proteins may exhibit other properties as disclosed herein.

Standard molecular biological and cloning techniques can be used to swap the equivalent domains between the zlmda24 polypeptide and those polypeptides to which they are fused. Generally, a DNA segment that encodes a domain of interest, e.g., zlmda24 helices A through D, or other domain described herein, is operably linked in frame to at least one other DNA segment encoding an additional polypeptide (for instance a domain or region from another cytokine, such as IL-10, or zcyto10, MDA7 or the like), and inserted into an appropriate expression vector, as described herein. Generally DNA constructs are made such that the several DNA segments that encode the corresponding regions of a polypeptide are operably linked in frame to make a single construct that encodes the entire fusion protein, or a functional portion thereof. For example, a DNA construct would encode from N-terminus to C-terminus a fusion protein comprising a signal polypeptide followed by a mature four helical bundle cytokine fusion protein containing helix A, followed by helix B, followed by helix C, followed by helix D. or for example, any of the above as interchanged with equivalent regions from another four helical bundle cytokine family protein. Such fusion proteins can be expressed, isolated, and assayed for activity as described herein. Moreover, such fusion proteins can be used to express and secrete fragments of the zlmda24 polypeptide, to be used, for example to inoculate an animal to generate anti-zlmda24 antibodies as described herein. For example a secretory signal sequence can be operably linked to Helix A, B, C, or D or a combination thereof (e.g., operably linked polypeptides comprising helices, A-B, B-C, C-D, A-C, B-D, or zlmda24 polypeptide fragments described herein), to secrete a fragment of zlmda24 polypeptide that can be purified as described herein and serve as an antigen to be inoculated into an animal to produce anti-zlmda24 antibodies, as described herein.

Zlmda24 polypeptides or fragments thereof may also be prepared through chemical synthesis. zlmda24 polypeptides may be monomers or multimers; glycosylated or non-glycosylated; pegylated or non-pegylated; and may or may not include an initial methionine amino acid residue. For example, the polypeptides can be prepared by solid phase peptide synthesis, for example as described by Merrifield, J. Am. Chem. Soc. 85:2149, 1963.

The activity of molecules of the present invention can be measured using a variety of assays that measure proliferation of and/or binding to cells expressing the zlmda24 receptor. Of particular interest are changes in zlmda24-dependent cells. Suitable cell lines to be engineered to be zlmda24-dependent include the IL-3-dependent BaF3 cell line (Palacios and Steinmetz, Cell 41: 727-734, 1985; Mathey-Prevot et al., Mol. Cell. Biol. 6: 4133-4135, 1986), FDC-P1 (Hapel et al., Blood 64: 786-790, 1984), and MO7e (Kiss et al., Leukemia 7: 235-240, 1993). Growth factor-dependent cell lines can be established according to published methods (e.g. Greenberger et al., Leukemia Res. 8: 363-375, 1984; Dexter et al., in Baum et al. Eds., Experimental Hematology Today, 8th Ann. Mtg. Int. Soc. Exp. Hematol. 1979, 145-156, 1980).

Proteins of the present invention are useful for stimulating proliferation, activation, differentiation and/or induction or inhibition of specialized cell function of cells of the involved homeostasis of the hematopoiesis and immune function. In particular, zlmda24 polypeptides are useful for stimulating proliferation, activation, differentiation, induction or inhibition of specialized cell functions of cells of the hematopoetic lineages, including, but not limited to, T cells, B cells, NK cells, dendritic cells, monocytes, and macrophages. Proliferation and/or differentiation of hematopoietic cells can be measured in vitro using cultured cells or in vivo by administering molecules of the present invention to the appropriate animal model. Assays measuring cell proliferation or differentiation are well known in the art. For example, assays measuring proliferation include such assays as chemosensitivity to neutral red dye (Cavanaugh et al., Investigational New Drugs 8:347-354, 1990, incorporated herein by reference), incorporation of radiolabelled nucleotides (Cook et al., Analytical Biochem. 179:1-7, 1989, incorporated herein by reference), incorporation of 5-bromo-2′-deoxyuridine (BrdU) in the DNA of proliferating cells (Porstmann et al., J. Immunol. Methods 82:169-179, 1985, incorporated herein by reference), and use of tetrazolium salts (Mosmann, J. Immunol. Methods 65:55-63, 1983; Alley et al., Cancer Res. 48:589-601, 1988; Marshall et al., Growth Reg. 5:69-84, 1995; and Scudiero et al., Cancer Res. 48:4827-4833, 1988; all incorporated herein by reference). Assays measuring differentiation include, for example, measuring cell-surface markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989; all incorporated herein by reference). Alternatively, zlmda24 polypeptide itself can serve as an additional cell-surface marker associated with stage-specific expression of a tissue. As such, direct measurement of zlmda24 polypeptide, or its loss of expression in a tissue as it differentiates, can serve as a marker for differentiation of testis tissue, or cells.

IL-10 is a cytokine that inhibits production of other cytokines, induces proliferation and differentiation of activated B lymphocytes, inhibits HIV-1 replication and exhibits antagonistic effects on gamma interferon. IL-10 appears to exist as a dimer formed from two alpha-helical polypeptide regions related by a 180° rotation. See, for example, Zdanov et al., Structure: 3(6): 591-601 (1996). IL-10 has been reported to be a product of activated Th2 T-cells, B-cells, keratinocytes and monocytes/macrophages that is capable of modulating a Th1 T-cell response. Such modulation may be accomplished by inhibiting cytokine synthesis by Th1 T-cells. See, for example, Hus et al., Int. Immunol. 4: 563 (1992) and D'Andrea et al., J. Exp. Med. 178: 1042 (1992). IL-10 has also been reported to inhibit cytokine synthesis by natural killer cells and monocytes/macrophages. See, for example, Hus et al. cited above and Fiorentino et al., J. Immunol. 146: 3444 (1991). In addition, IL-10 has been found to have a protective effect with respect to insulin dependent diabetes mellitus. Similarly, as a cytokine-like molecule sharing polypeptide structure with IL-10, zlmda24 can have these above disclosed activities, and the assays used to assess IL-10 activity can be applied to assay zlmda24 activity.

The molecules of the present invention can be assayed in vivo using viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acid (for review, see T. C. Becker et al., Meth. Cell Biol. 43:161-89, 1994; and J. T. Douglas and D. T. Curiel, Science & Medicine 4:44-53, 1997). The adenovirus system offers several advantages: (i) adenovirus can accommodate relatively large DNA inserts; (ii) can be grown to high-titer; (iii) infect a broad range of mammalian cell types; and (iv) can be used with many different promoters including ubiquitous, tissue specific, and regulatable promoters. Also, because adenoviruses are stable in the bloodstream, they can be administered by intravenous injection.

Using adenovirus vectors where portions of the adenovirus genome are deleted, inserts are incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293 cells can be grown as adherent cells or in suspension culture at relatively high cell density to produce significant amounts of protein (See Gamier et al., Cytotechnol. 15:145-55, 1994). With either protocol, an expressed, secreted heterologous protein can be repeatedly isolated from the cell culture supernatant, lysate, or membrane fractions depending on the disposition of the expressed protein in the cell. Within the infected 293 cell production protocol, non-secreted proteins may also be effectively obtained.

Zlmda24 is specifically expressed a tissue that contracts. For example contractile tissues in which zlmda24 is expressed include tissues in testis, e.g., vas deferens; and may be expressed in salivary gland and affect gastrointestinal tissues, e.g., colon and small intestine. The effects of zlmda24 polypeptide, its antagonists and agonists, on tissue contractility can be measured in vitro using a tensiometer with or without electrical field stimulation. Such assays are known in the art and can be applied to tissue samples, such as aortic rings, vas deferens, ilium, uterine and other contractile tissue samples, as well as to organ systems, such as atria, and can be used to determine whether zlmda24 polypeptide, its agonists or antagonists, enhance or depress contractility. Molecules of the present invention are hence useful for treating dysfunction associated with contractile tissues or can be used to suppress or enhance contractility in vivo. As such, molecules of the present invention have utility in treating cardiovascular disease, infertility, in vitro fertilization, birth control, treating impotence or other male reproductive dysfunction, as well as inducing birth.

The effect of the zlmda24 polypeptides, antagonists and agonists of the present invention on contractility of tissues including testis, gastrointestinal tissues, and salivary gland can be measured in a tensiometer that measures contractility and relaxation in tissues. See, Dainty et al., J. Pharmacol. 100:767, 1990; Rhee et al., Neurotox. 16: 179, 1995; Anderson, M. B., Endocrinol. 114:364-368, 1984; and Downing, S. J. and Sherwood, O. D, Endocrinol. 116:1206-1214, 1985. For example, measuring vasodilatation of aortic rings is well known in the art. Briefly, aortic rings are taken from 4 month old Sprague Dawley rats and placed in a buffer solution, such as modified Krebs solution (118.5 mM NaCl, 4.6 mM KCl, 1.2 mM MgSO₄.7H₂O, 1.2 mM KH₂PO₄, 2.5 mM CaCl₂.2H₂O, 24.8 mM NaHCO₃ and 10 mM glucose). One of skill in the art would recognize that this method can be used with other animals, such as rabbits, other rat strains, Guinea pigs, and the like. The rings are then attached to an isometric force transducer (Radnoti Inc., Monrovia, Calif.) and the data recorded with a Ponemah physiology platform (Gould Instrument systems, Inc., Valley View, Ohio) and placed in an oxygenated (95% O₂, 5% CO₂) tissue bath containing the buffer solution. The tissues are adjusted to 1 gram resting tension and allowed to stabilize for about one hour before testing. The integrity of the rings can be tested with norepinepherin (Sigma Co., St. Louis, Mo.) and Carbachol, a muscarinic acetylcholine agonist (Sigma Co.). After integrity is checked, the rings are washed three times with fresh buffer and allowed to rest for about one hour. To test a sample for vasodilatation, or relaxation of the aortic ring tissue, the rings are contracted to two grams tension and allowed to stabilize for fifteen minutes. A zlmda24 polypeptide sample is then added to 1, 2 or 3 of the 4 baths, without flushing, and tension on the rings recorded and compared to the control rings containing buffer only. Enhancement or relaxation of contractility by zlmda24 polypeptides, their agonists and antagonists is directly measured by this method, and it can be applied to other contractile tissues such as testis.

The activity of molecules of the present invention can be measured using a variety of assays that measure stimulation of gastrointestinal cell contractility, modulation of nutrient uptake and/or secretion of digestive enzymes. Of particular interest are changes in contractility of smooth muscle cells. For example, the contractile response of segments of mammalian duodenum or other gastrointestinal smooth muscles tissue (Depoortere et al., J. Gastrointestinal Motility 1:150-159, 1989, incorporated herein by reference). An exemplary in vivo assay uses an ultrasonic micrometer to measure the dimensional changes radially between commissures and longitudinally to the plane of the valve base (Hansen et al., Society of Thoracic Surgeons 60:S384-390, 1995).

Gastric motility is generally measured in the clinical setting as the time required for gastric emptying and subsequent transit time through the gastrointestinal tract. Gastric emptying scans are well known to those skilled in the art, and briefly, comprise use of an oral contrast agent, such as barium, or a radiolabeled meal. Solids and liquids can be measured independently. A test food or liquid is radiolabeled with an isotope (e.g. ^{99m}Tc), and after ingestion or administration, transit time through the gastrointestinal tract and gastric emptying are measured by visualization using gamma cameras (Meyer et al., Am. J. Dig. Dis. 21:296, 1976; Collins et al., Gut 24:1117, 1983; Maughan et al., Diabet. Med. 13 9 Supp. 5:S6-10, 1996 and Horowitz et al., Arch. Intern. Med. 145:1467-1472, 1985). These studies may be performed before and after the administration of a promotility agent to quantify the efficacy of the drug.

The tissue specificity of zlmda24 expression suggests a role in spermatogenesis, a process that is remarkably similar to the development of blood cells (hematopoiesis). Briefly, spermatogonia undergo a maturation process similar to the differentiation of hematopoietic stem cells. In view of the tissue specificity observed for zlmda24, agonists and antagonists have enormous potential in both in vitro and in vivo applications. Zlmda24 polypeptides, anti-zlmda24 antibodies, agonists and antagonists may also prove useful in modulating (or regulating) spermatogenesis, and/or induction or inhibition of specialized cell function of cells involved in spermatogenesis, fertility or sperm capacitation, and thus aid in overcoming infertility or have use in preventing conception. Antagonists are useful as research reagents for characterizing sites of ligand-receptor interaction. In vivo, zlmda24 polypeptides, agonists or antagonists may find application in the treatment of male infertility or as a male contraceptive agents.

The zlmda24 polypeptides, antagonists of agonists, of the present invention can also modulate sperm capacitation. Before reaching the oocyte or egg and initiating an egg-sperm interaction, the sperm must be activated. The sperm undergo a gradual capacitation, lasting up to 3 or 4 hours in vitro, during which the plasma membrane of the sperm head and the outer acrosomal membrane fuse to form vesicles that facilitate the release of acrosomal enzymes. The acrosomal membrane surrounds the acrosome or acrosomal cap which is located at the anterior end of the nucleus in the sperm head. In order for the sperm to fertilize egg the sperm must penetrate the oocyte. To enable this process the sperm must undergo acrosomal exocytosis, also known as the acrosomal reaction, and release the acrosomal enzymes in the vicinity of the oocyte. These enzymes enable the sperm to penetrate the various oocyte layers, (the cumulus oophorus, the corona radiata and the zona pellucida). The released acrosomal enzymes include hyaluronidase and proacrosin, in addition to other enzymes such as proteases. During the acrosomal reaction, proacrosin is converted to acrosin, the active form of the enzyme, which is required for and must occur before binding and penetration of the zona pellucida is possible. A combination of the acrosomal lytic enzymes and sperm tail movements allow the sperm to penetrate the oocyte layers. Numerous sperm must reach the egg and release acrosomal enzymes before the egg can finally be fertilized. Only one sperm will successfully bind to, penetrate and fertilize the egg, after which the zona hardens so that no other sperm can penetrate the egg (Zaneveld, in Male Infertility Chapter 11, Comhaire (Ed.), Chapman & Hall, London, 1996). Peptide hormones, such as insulin homologs are associated with sperm activation and egg-sperm interaction. For instance, capacitated sperm incubated with relaxin show an increased percentage of progressively motile sperm, increased zona penetration rates, and increased percentage of viable acrosome-reacted sperm (Carrell et al., Endocr. Res. 21:697-707, 1995). Similarity of the zlmda24 polypeptide structure with peptide hormones and localization of Zlmda24 to the testis suggests that the zlmda24 polypeptides described herein play a role in these and other reproductive processes.

Accordingly, proteins of the present invention can have applications in enhancing fertilization during assisted reproduction in humans and in animals. Such assisted reproduction methods are known in the art and include artificial insemination, in vitro fertilization, embryo transfer and gamete intrafallopian transfer. Such methods are useful for assisting men and women who have physiological or metabolic disorders preventing natural conception or can be used to enhance in vitro fertilization. Such methods are also used in animal breeding programs, such as for livestock breeding and could be used as methods for the creation of transgenic animals. Proteins of the present invention can be combined with sperm, an egg or an egg-sperm mixture prior to fertilization of the egg. In some species, sperm capacitate spontaneously during in vitro fertilization procedures, but normally sperm capacitate over an extended period of time both in vivo and in vitro. It is advantageous to increase sperm activation during such procedures to enhance the likelihood of successful fertilization. The washed sperm or sperm removed from the seminal plasma used in such assisted reproduction methods has been shown to have altered reproductive functions, in particular, reduced motility and zona interaction. To enhance fertilization during assisted reproduction methods sperm is capacitated using exogenously added compounds. Suspension of the sperm in seminal plasma from normal subjects or in a “capacitation media” containing a cocktail of compounds known to activate sperm, such as caffeine, dibutyl cyclic adenosine monophosphate (dbcAMP) or theophylline, have resulted in improved reproductive function of the sperm, in particular, sperm motility and zonae penetration (Park et al., Am. J. Obstet. Gynecol. 158:974-9, 1988; Vandevoort et al., Mol. Repro. Develop. 37:299-304, 1993; Vandevoort and Overstreet, J. Androl. 16:327-33, 1995). The presence of immunoreactive relaxin in vivo and in association with cryopreserved semen, was shown to significantly increase sperm motility (Juang et al., Anim. Reprod. Sci. 20:21-9, 1989; Juang et al., Anim. Reprod. Sci. 22:47-53, 1990). Porcine relaxin stimulated sperm motility in cryopreserved human sperm (Colon et al., Fertil. Steril. 46:1133-39, 1986; Lessing et al., Fertil. Steril. 44:406-9, 1985) and preserved ability of washed human sperm to penetrate cervical mucus in vitro (Brenner et al., Fertil. Steril. 25 42:92-6, 1984). Polypeptides of the present invention can used in such methods to enhance viability of cryopreserved sperm, enhance sperm motility and enhance fertilization, particularly in association with methods of assisted reproduction.

In cases where pregnancy is not desired, zlmda24 polypeptide or polypeptide fragments may function as germ-cell-specific antigens for use as components in “immunocontraceptive” or “anti-fertility” vaccines to induce formation of anti-zlmda24 antibodies and/or cell mediated immunity to selectively inhibit a process, or processes, critical to successful reproduction in humans and animals. The use of sperm and testis antigens in the development of immunocontraceptives have been described (O'Hern et al., Biol Reprod. 52:311-39, 1995; Diekman and Herr, Am. J. Reprod. Immunol. 37:111-17, 1997; Zhu and Naz, Proc. Natl. Acad. Sci. USA 94:4704-9,1997). A vaccine based on human chorionic gonadotrophin (HCG) linked to a diphtheria or tetanus carrier was in clinical trials (Talwar et al., Proc. Natl. Acad. Sci. USA 91:8532-36, 1994). A single injection resulted in production of high titer antibodies that persisted for nearly a year in rabbits (Stevens, Am. J. Reprod. Immunol. 29:176-88, 1993). Such methods of immunocontraception using vaccines would include a zlmda24 testes-specific protein or fragment thereof. The Zlmda24 protein or fragments can be conjugated to a carrier protein or peptide, such as tetanus or diphtheria toxoid. An adjuvant, as described above, can be included and the protein or fragment can be noncovalently associated with other molecules to enhance intrinsic immunoreactivity. Methods for administration and methods for determining the number of administrations are known in the art. Such a method might include a number of primary injections over several weeks followed by booster injections as needed to maintain a suitable antibody titer.

Regulation of reproductive function in males and females is controlled in part by feedback inhibition of the hypothalamus and anterior pituitary by blood-borne hormones. Testis proteins, such as activins and inhibins, have been shown to regulate secretion of active molecules including follicle stimulating hormone (FSH) from the pituitary (Ying, Endodcr. Rev. 9:267-93, 1988; Plant et al., Hum. Reprod. 8:41-44,1993). Inhibins, also expressed in the ovaries, have been shown to regulate ovarian functions (Woodruff et al., Endocr. 132:2332-42,1993; Russell et al., J. Reprod. Fertil. 100:115-22, 1994). Relaxin has been shown to be a systemic and local acting hormone regulating follicular and uterine growth (Bagnell et al., J. Reprod. Fertil. 48:127-38, 1993). As such, the polypeptides of the present invention may also have effects on female gametes and reproductive tract. These functions may also be associated with zlmda24 polypeptides and may be used to regulate testicular or ovarian functions.

As a ligand, the activity of zlmda24 polypeptide can be measured by a silicon-based biosensor microphysiometer which measures the extracellular acidification rate or proton excretion associated with receptor binding and subsequent physiologic cellular responses. An exemplary device is the Cytosensor™ Microphysiometer manufactured by Molecular Devices, Sunnyvale, Calif. A variety of cellular responses, such as cell proliferation, ion transport, energy production, inflammatory response, regulatory and receptor activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including zlmda24 polypeptide, its agonists, or antagonists. Preferably, the microphysiometer is used to measure responses of a zlmda24-responsive eukaryotic cell, compared to a control eukaryotic cell that does not respond to zlmda24 polypeptide. Zlmda24-responsive eukaryotic cells comprise cells into which a receptor for zlmda24 has been transfected creating a cell that is responsive to zlmda24; or cells naturally responsive to zlmda24 such as cells derived from testis tissues. Differences, measured by a change, for example, an increase or diminution in extracellular acidification, in the response of cells exposed to zlmda24 polypeptide, relative to a control not exposed to zlmda24, are a direct measurement of zlmda24-modulated cellular responses. Moreover, such zlmda24-modulated responses can be assayed under a variety of stimuli. Using the microphysiometer, there is provided a method of identifying agonists of zlmda24 polypeptide, comprising providing cells responsive to a zlmda24 polypeptide, culturing a first portion of the cells in the absence of a test compound, culturing a second portion of the cells in the presence of a test compound, and detecting a change, for example, an increase or diminution, in a cellular response of the second portion of the cells as compared to the first portion of the cells. The change in cellular response is shown as a measurable change extracellular acidification rate. Moreover, culturing a third portion of the cells in the presence of zlmda24 polypeptide and the absence of a test compound can be used as a positive control for the zlmda24-responsive cells, and as a control to compare the agonist activity of a test compound with that of the zlmda24 polypeptide. Moreover, using the microphysiometer, there is provided a method of identifying antagonists of zlmda24 polypeptide, comprising providing cells responsive to a zlmda24 polypeptide, culturing a first portion of the cells in the presence of zlmda24 and the absence of a test compound, culturing a second portion of the cells in the presence of zlmda24 and the presence of a test compound, and detecting a change, for example, an increase or a diminution in a cellular response of the second portion of the cells as compared to the first portion of the cells. The change in cellular response is shown as a measurable change extracellular acidification rate. Antagonists and agonists, for zlmda24 polypeptide, can be rapidly identified using this method.

Moreover, zlmda24 can be used to identify cells, tissues, or cell lines which respond to a zlmda24-stimulated pathway. The microphysiometer, described above, can be used to rapidly identify ligand-responsive cells, such as cells responsive to zlmda24 of the present invention. Cells can be cultured in the presence or absence of zlmda24 polypeptide. Those cells which elicit a measurable change in extracellular acidification in the presence of zlmda24 are responsive to zlmda24. Such cell lines, can be used to identify antagonists and agonists of zlmda24 polypeptide as described above.

In view of the tissue distribution observed for zlmda24 receptor agonists (including the natural ligand/substrate/cofactor/etc.) and/or antagonists have enormous potential in both in vitro and in vivo applications. Compounds identified as zlmda24 agonists are useful for expansion, proliferation, activation, differentiation, and/or induction or inhibition of specialized cell functions of cells involved in homeostasis of hematopoiesis and immune function, and/or induction or inhibition of specialized cell function of cells involved in spermatogenesis, fertility, or sperm capacitation. For example, zlmda24 and agonist compounds are useful as components of defined cell culture media, and may be used alone or in combination with other cytokines and hormones to replace serum that is commonly used in cell culture. Agonists are thus useful in specifically promoting the growth and/or development of T-cells, B-cells, and other cells of the lymphoid and myeloid lineages in culture.

Antagonists are also useful as research reagents for characterizing sites of ligand-receptor interaction. Antagonists are useful to inhibit expansion, proliferation, activation, and/or differentiation of cells involved in regulating hematopoiesis. Inhibitors of zlmda24 activity (zlmda24 antagonists) include anti-zlmda24 antibodies and soluble zlmda24 receptors, as well as other peptidic and non-peptidic agents (including ribozymes).

Zlmda24 can also be used to identify inhibitors (antagonists) of its activity. Test compounds are added to the assays disclosed herein to identify compounds that inhibit the activity of zlmda24. In addition to those assays disclosed herein, samples can be tested for inhibition of zlmda24 activity within a variety of assays designed to measure receptor binding, the stimulation/inhibition of zlmda24-dependent cellular responses or proliferation of zlmda24 receptor-expressing cells.

A zlmda24 polypeptide can be expressed as a fusion with an immunoglobulin heavy chain constant region, typically an Fc fragment, which contains two constant region domains and lacks the variable region. Methods for preparing such fusions are disclosed in U.S. Pat. Nos. 5,155,027 and 5,567,584. Such fusions are typically secreted as multimeric molecules wherein the Fc portions are disulfide bonded to each other and two non-Ig polypeptides are arrayed in closed proximity to each other. Fusions of this type can be used to (e.g., for dimerization, increasing stability and in vivo half-life, affinity purify ligand, in vitro assay tool, antagonist). For use in assays, the chimeras are bound to a support via the Fc region and used in an ELISA format.

Polypeptides containing the receptor-binding region of the ligand can be used for purification of receptor. The ligand polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting media will generally be configured in the form of a column, and fluids containing receptors are passed through the column one or more times to allow receptor to bind to the ligand polypeptide. The receptor is then eluted using changes in salt concentration, chaotropic agents (MnCl₂), or pH to disrupt ligand-receptor binding.

zlmda24 polypeptides or zlmda24 fusion proteins are used, for example, to identify the zlmda24 receptor. Using labeled zlmda24 polypeptides, cells expressing the receptor are identified by fluorescence immunocytometry or immunohistochemistry. zlmda24 polypeptides are useful in determining the distribution of the receptor on tissues or specific cell lineages, and to provide insight into receptor/ligand biology. An exemplary method to identify a zlmda24 receptor in vivo or in vitro, e.g., in cell lines, is to us a zlmda24 polypeptide fused to the catalytic domain of Alkaline phosphatase (AP), as described in Feiner, L. et al., Neuron 19:539-545, 1997. Such AP fusions, as well as radiolabeled zlmda24, zlmda24 fusions with fluorescent lables, and others described herein, combined with standard cloning techniques enable one of skill in the art to visualize, identify and clone the zlmda24 receptor.

Conversely, a zlmda24-binding polypeptide can be used for purification of ligand. The polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing ligand are passed through the column one or more times to allow ligand to bind to the receptor polypeptide. The ligand is then eluted using changes in salt concentration, chaotropic agents (guanidine HCl), or pH to disrupt ligand-receptor binding.

An assay system that uses a ligand-binding receptor (or an antibody, one member of a complement/ anti-complement pair) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding. Alternatively, ligand/receptor binding can be analyzed using SELDI™ technology (Ciphergen, Inc., Palo Alto, Calif.).

Ligand-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

Zlmda24 polypeptides can also be used to prepare antibodies that bind to zlmda24 epitopes, peptides or polypeptides. The zlmda24 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a zlmda24 polypeptide (e.g., SEQ ID NO:2). Polypeptides comprising a larger portion of a zlmda24 polypeptide, i.e., from 30 to 100 residues up to the entire length of the amino acid sequence are included. Antigens or immunogenic epitopes can also include attached tags, adjuvants and carriers, as described herein. Suitable antigens include the human zlmda24 polypeptide encoded by SEQ ID NO:2 from amino acid number 32 to amino acid number 253, or a contiguous 9 to 222 amino acid fragment thereof. Suitable antigens also include the mouse zlmda24 polypeptide encoded by SEQ ID NO:4 from amino acid number 28 to amino acid number 253, or a contiguous 9 to 226 amino acid fragment thereof. Other suitable antigens include helices A-D of the four-helical-bundle structure, individually or operably linked in combination, as described herein. Preferred peptides to use as antigens are hydrophilic peptides such as those predicted by one of skill in the art from a hydrophobicity plot, as described herein. For example suitable hydrophilic peptides include: (1) amino acid number 34 (Gln) to amino acid number 39 (Arg) of SEQ ID NO:2; (2) amino acid number 59 (Asn) to amino acid number 64 (Asp) of SEQ ID NO:2; (3) amino acid number 58 (Ala) to amino acid number 63 (Lys) of SEQ ID NO:2; and (4) amino acid number 116 (Gly) to amino acid number 121 (Glu) of SEQ ID NO:2; and (1) amino acid number 34 (Gln) to amino acid number 39 (Arg) of SEQ ID NO:4; (2) amino acid number 59 (Asn) to amino acid number 64 (Asp) of SEQ ID NO:4; (3) amino acid number 159 (Asp) to amino acid number 164 (Asp) of SEQ ID NO:4; and (4) amino acid number 118 (Glu) to amino acid number 123 (Glu) of SEQ ID NO:4. Moreover, zlmda24 antigenic epitopes as predicted by a Jameson-Wolf plot, e.g., using DNASTAR Protean program (DNASTAR, Inc., Madison, Wis.) serve as preferred antigens, including polypeptides comprising: (1) amino acid number 7 (Asn) to amino acid number 16 (Pro) of SEQ ID NO:2; (2) amino acid number 60 (Val) to amino acid number 67 (Asp) of SEQ ID NO:2; (3) amino acid number 101 (Glu) to amino acid number 107 (Leu) of SEQ ID NO:2; (4) amino acid number 162 (Thr) to amino acid number 169 (Glu) of SEQ ID NO:2; (5) amino acid number 194 (Lys) to amino acid number 200 (Leu) of SEQ ID NO:2; (6) amino acid number 218 (Cys) to amino acid number 225 (Asp) of SEQ ID NO:2; (7) amino acid number Ala (249) to amino acid number 252 (Arg) of SEQ ID NO:2; and (1) amino acid number 6 (His) to amino acid number 14 (Gln) of SEQ ID NO:4; (2) amino acid number 59 (Asn) to amino acid number 67 (Glu) of SEQ ID NO:4; (3) amino acid number 101 (Glu) to amino acid number 106 (Thr) of SEQ ID NO:4; (4) amino acid number 159 (Asp) to amino acid number 166 (Ser) of SEQ ID NO:4; (5) amino acid number 158 (Leu) to amino acid number 167 (Ile) of SEQ ID NO:4; (6) amino acid number 193 (Ala) to amino acid number 199 (Asn) of SEQ ID NO:4; (7) amino acid number Ala (249) to amino acid number 252 (Arg) of SEQ ID NO:4.

Antibodies from an immune response generated by inoculation of an animal with these antigens (or immunogens) can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a zlmda24 polypeptide or a fragment thereof. The immunogenicity of a zlmda24 polypeptide may be increased through the use of an adjuvant, such as alum (aluminum hydroxide) or Freund's complete or incomplete adjuvant. Polypeptides useful for immunization also include fusion polypeptides, such as fusions of zlmda24 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Moreover, human antibodies can be produced in transgenic, non-human animals that have been engineered to contain human immunoglobulin genes as disclosed in WIPO Publication WO 98/24893. It is preferred that the endogenous immunoglobulin genes in these animals be inactivated or eliminated, such as by homologous recombination.

Antibodies are considered to be specifically binding if: 1) they exhibit a threshold level of binding activity, and 2) they do not significantly cross-react with related polypeptide molecules. A threshold level of binding is determined if anti-zlmda24 antibodies herein bind to a zlmda24 polypeptide, peptide or epitope with an affinity at least 10-fold greater than the binding affinity to control (non-zlmda24) polypeptide. It is preferred that the antibodies exhibit a binding affinity (Kₐ) of 10⁶ M⁻¹ or greater, preferably 10⁷ M⁻¹ or greater, more preferably 10 8 M ¹ or greater, and most preferably 10⁹ M⁻¹ or greater. The binding affinity of an antibody can be readily determined by one of ordinary skill in the art, for example, by Scatchard analysis (Scatchard, G., Ann. NY Acad. Sci. 51: 660-672, 1949).

Whether anti-zlmda24 antibodies do not significantly cross-react with related polypeptide molecules is shown, for example, by the antibody detecting zlmda24 polypeptide but not known related polypeptides using a standard Western blot analysis (Ausubel et al., ibid.). Examples of known related polypeptides are those disclosed in the prior art, such as known orthologs, and paralogs, and similar known members of a protein family. Screening can also be done using non-human zlmda24, and zlmda24 mutant polypeptides. Moreover, antibodies can be “screened against” known related polypeptides, to isolate a population that specifically binds to the zlmda24 polypeptides. For example, antibodies raised to zlmda24 are adsorbed to related polypeptides adhered to insoluble matrix; antibodies specific to zlmda24 will flow through the matrix under the proper buffer conditions. Screening allows isolation of polyclonal and monoclonal antibodies non-crossreactive to known closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental Immunology, Paul (eds.), Raven Press, 1993; Getzoff et al., Adv. in Immunol 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984. Specifically binding anti-zlmda24 antibodies can be detected by a number of methods in the art, and disclosed below.

A variety of assays known to those skilled in the art can be utilized to detect antibodies that bind to zlmda24 proteins or polypeptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant zlmda24 protein or polypeptide.

Alternative techniques for generating or selecting antibodies useful herein include in vitro exposure of lymphocytes to zlmda24 protein or peptide, and selection of antibody display libraries in phage or similar vectors (for instance, through use of immobilized or labeled zlmda24 protein or peptide). Genes encoding polypeptides having potential zlmda24 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides that interact with a known target, which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the zlmda24 sequences disclosed herein to identify polypeptides that bind to zlmda24. These “binding polypeptides” which interact with zlmda24 polypeptides can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and neutralizing activity, e.g., for blocking interaction between ligand and receptor, or viral binding to a receptor. The binding polypeptides can also be used for diagnostic assays for determining circulating levels of zlmda24 polypeptides; for detecting or quantitating soluble zlmda24 polypeptides as marker of underlying pathology or disease. These binding polypeptides can also act as zlmda24 “antagonists” to block zlmda24 binding and signal transduction in vitro and in vivo. These anti-zlmda24 binding polypeptides would be useful for inhibiting zlmda24 activity or protein-binding.

Antibodies to zlmda24 may be used for tagging cells that express zlmda24; for isolating zlmda24 by affinity purification; for diagnostic assays for determining circulating levels of zlmda24 polypeptides; for detecting or quantitating soluble zlmda24 as a marker of underlying pathology or disease; in analytical methods employing FACS; for screening expression libraries; for generating anti-idiotypic antibodies; and as neutralizing antibodies or as antagonists to block zlmda24 activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to zlmda24 or fragments thereof may be used in vitro to detect denatured zlmda24 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zlmda24 polypeptides or anti-zlmda24 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the polypeptide or antibody portion. For these purposes, biotin/streptavidin is an exemplary complementary/anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, if the polypeptide has multiple functional domains (i.e., an activation domain or a receptor binding domain, plus a targeting domain), a fusion protein including only the targeting domain may be suitable for directing a detectable molecule, a cytotoxic molecule or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/ cytotoxic molecule conjugates. As zlmda24 expression is testis-specific, such uses in detection and targeting of cytotoxic molecules to testicular cancer, and use of such anti-complementary molecules in identification, diagnosis and treatment of testicular tissues are evident.

In another embodiment, zlmda24 cytokine fusion proteins or antibody-cytokine fusion proteins can be used for enhancing in vivo killing of target tissues (for example, testicular or lymphoid cancers), if the zlmda24 polypeptide or anti-zlmda24 antibody targets the hyperproliferative cell (See, generally, Homick et al., Blood 89:4437-47, 1997). The described fusion proteins enable targeting of a cytokine to a desired site of action, thereby providing an elevated local concentration of cytokine. Suitable zlmda24 polypeptides or anti-zlmda24 antibodies target an undesirable cell or tissue (i.e., a tumor or a leukemia), and the fused cytokine mediated improved target cell lysis by effector cells. Suitable cytokines for this purpose include interleukin 2 and granulocyte-macrophage colony-stimulating factor (GM-CSF), for instance.

In yet another embodiment, if the zlmda24 polypeptide or anti-zlmda24 antibody targets testicular or vascular cells or tissues, such polypeptide or antibody may be conjugated with a radionuclide, and particularly with a beta-emitting radionuclide, to reduce restenosis. Such therapeutic approaches pose less danger to clinicians who administer the radioactive therapy. For instance, iridium-192 impregnated ribbons placed into stented vessels of patients until the required radiation dose was delivered showed decreased tissue growth in the vessel and greater luminal diameter than the control group, which received placebo ribbons. Further, revascularisation and stent thrombosis were significantly lower in the treatment group. Similar results are predicted with targeting of a bioactive conjugate containing a radionuclide, as described herein.

The bioactive polypeptide or antibody conjugates described herein can be delivered intravenously, intraarterially or intraductally, or may be introduced locally at the intended site of action.

Zlmda24 has a cytokine-like structure and hence may modulate immunological function and play a role in the immune system. Thus, the polypeptides of the present invention may have an effect on the growth/expansion and/or differentiated state of T- or B-Cells, T- or B-cell progenitors, NK cells or NK progenitors. Moreover, zlmda24 can effect proliferation and/or differentiation of T cells and B cells in vivo. Factors that both stimulate proliferation of hematopoietic progenitors and activate mature cells are generally known. NK cells are responsive to IL-2 alone, but proliferation and activation generally require additional growth factors. For example, it has been shown that IL-7 and Steel Factor (c-kit ligand) were required for colony formation of NK progenitors. L-15 +IL-2 in combination with IL-7 and Steel Factor was more effective (Mrózek et al., Blood 87:2632-2640, 1996). However, unidentified cytokines may be necessary for proliferation of specific subsets of NK cells and/or NK progenitors (Robertson et. al., Blood 76:2451-2438, 1990). A composition comprising zlmda24 and L- 15 may stimulate NK progenitors and NK cells, as a composition that is more potent than previously described factors and combinations of factors. Similarly, such combinations of factors that include zlmda24 may also affect other hematopoietic and lymphoid cell types, such as T-cells, B-cells, macrophages, dendritic cells, and the like.

Most four-helix bundle cytokines as well as other proteins produced by activated lymphocytes play an important biological role in cell differentiation, activation, recruitment and homeostasis of cells throughout the body. Therapeutic utility includes treatment of diseases that require immune regulation including autoimmune diseases, such as, rheumatoid arthritis, multiple sclerosis, myasthenia gravis, systemic lupus erythomatosis (SLE) and diabetes. Zlmda24 may be important in the regulation of inflammation, and therefore would be useful in treating rheumatoid arthritis, asthma, ulcerative colitis, inflammatory bowel disease, Crohn's disease, and sepsis. There may be a role of zlmda24 in mediating tumorgenesis, and therefore would be useful in the treatment of cancer. Zlmda24 may be a potential therapeutic in suppressing the immune system which would be important for reducing graft rejection. Zlmda24 may have usefulness in prevention of graft-vs-host disease.

The proteins of the present invention can also be used ex vivo, such as in autologous marrow culture. Briefly, bone marrow is removed from a patient prior to chemotherapy or organ transplant and treated with zlmda24, optionally in combination with one or more other cytokines. The treated marrow is then returned to the patient after chemotherapy to speed the recovery of the marrow or after transplant to suppress graft vs. Host disease. In addition, the proteins of the present invention can also be used for the ex vivo expansion of marrow or peripheral blood progenitor (PBPC) cells.

Prior to treatment, marrow can be stimulated with stem cell factor (SCF) to release early progenitor cells into peripheral circulation. These progenitors can be collected and concentrated from peripheral blood and then treated in culture with zlmda24, optionally in combination with one or more other cytokines, including but not limited to IL-10, zcyto10, MDA7, SCF, IL-2, IL-4, IL-7 or IL-15, to differentiate and proliferate into high-density lymphoid cultures, which can then be returned to the patient following chemotherapy or transplantation.

Alternatively, zlmda24 may activate the immune system, which would be important in boosting immunity to infectious diseases, treating immunocompromised patients, such as HIV+patients, or in improving vaccines. In particular, zlmda24 stimulation or expansion of T-cells, B-cells, NK cells, and the like, or their progenitors, would provide therapeutic value in treatment of viral infection, and as an anti-neoplastic factor. NK cells are thought to play a major role in elimination of metastatic tumor cells and patients with both metastases and solid tumors have decreased levels of NK cell activity (Whiteside et. al., Curr. Top. Microbiol. Immunol. 230:221-244, 1998).

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products, and receptors. The stage of a cell population's differentiation is monitored by identification of markers present in the cell population. Myocytes, osteoblasts, adipocytes, chrondrocytes, fibroblasts and reticular cells are believed to originate from a common mesenchymal stem cell (Owen et al., Ciba Fdn. Symp. 136:42-46, 1988). Markers for mesenchymal stem cells have not been well identified (Owen et al., J. of Cell Sci. 87:731-738, 1987), so identification is usually made at the progenitor and mature cell stages. The novel polypeptides of the present invention may be useful for studies to isolate mesenchymal stem cells and myocyte or other progenitor cells, both in vivo and ex vivo.

There is evidence to suggest that factors that stimulate specific cell types down a pathway towards terminal differentiation or dedifferentiation affect the entire cell population originating from a common precursor or stem cell. Assays measuring differentiation include, for example, measuring cell markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989; all incorporated herein by reference). Alternatively, zlmda24 polypeptide itself can serve as an additional cell-surface or secreted marker associated with stage-specific expression of a tissue, such as testis tissue. As such, direct measurement of zlmda24 polypeptide, or its loss of expression in a tissue as it differentiates, can serve as a marker for differentiation of tissues.

Similarly, direct measurement of zlmda24 polypeptide, or its loss of expression in a tissue can be determined in a tissue or cells as they undergo tumor progression. Increases in invasiveness and motility of cells, or the gain or loss of expression of zlmda24 in a pre-cancerous or cancerous condition, in comparison to normal tissue, can serve as a diagnostic for transformation, invasion and metastasis in tumor progression. As such, knowledge of a tumor's stage of progression or metastasis will aid the physician in choosing the most proper therapy, or aggressiveness of treatment, for a given individual cancer patient. Methods of measuring gain and loss of expression (of either mRNA or protein) are well known in the art and described herein and can be applied to zlmda24 expression. For example, appearance or disappearance of polypeptides that regulate cell motility can be used to aid diagnosis and prognosis of prostate cancer (Banyard, J. and Zetter, B. R., Cancer and Metast. Rev. 17:449-458, 1999). As an effector of cell motility, or as a testis-specific marker, zlmda24 gain or loss of expression may serve as a diagnostic for testicular, lymphoid, B-cell, endothelial, hematopoietic and other cancers. Moreover, analogous to the prostate specific antigen (PSA), as a naturally-expressed testicular marker, increased levels of zlmda24 polypeptides, or anti-zlmda24 antibodies in a patient, relative to a normal control can be indicative of testicular disease, such as testicular cancer (See, e.g., Mulders, T M T, et al., Eur. J. Surgical Oncol. 16:37-41, 1990). Moreover, as zlmda24 expression appears to be restricted to testis in normal human tissues, lack of zlmda24 expression in testis or strong zlmda24 expression in non-testis tissue would serve as a diagnostic of an abnormality in the cell or tissue type, of invasion or metastasis of cancerous testicular tissues into non-testicular tissue, and could aid a physician in directing further testing or investigation, or aid in directing therapy.

In addition, as zlmda24 is testis-specific, polynucleotide probes, anti-zlmda24 antibodies, and detection the presence of zlmda24 polypeptides in tissue can be used to assess whether testis tissue is present, for example, after surgery involving the excision of a diseased or cancerous testis. As such, the polynucleotides, polypeptides, and antibodies of the present invention can be used as an aid to determine whether all testis tissue is excised after surgery, for example, after surgery for testis cancer. In such instances, it is especially important to remove all potentially diseased tissue to maximize recovery from the cancer, and to minimize recurrence. Preferred embodiments include fluorescent, radiolabeled, or calorimetrically labeled anti-zlmda24 antibodies and zlmda24 polypeptide binding partners, that can be used histologically or in situ.

Moreover, the activity and effect of zlmda24 on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Appropriate tumor models for our studies include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly MS, et al. Cell 79: 315-328,1994). C57BL6/J mice are treated with an experimental agent either through daily injection of recombinant protein, agonist or antagonist or a one-time injection of recombinant adenovirus. Three days following this treatment, 10⁵ to 10⁶ cells are implanted under the dorsal skin. Alternatively, the cells themselves may be infected with recombinant adenovirus, such as one expressing zlmda24, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500-1800 mm³ in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., zlmda24, on the ability of the tumor to recruit vasculature and undergo metastasis can thus be assessed. In addition, aside from using adenovirus, the implanted cells can be transiently transfected with zlmda24. Use of stable zlmda24 transfectants as well as use of induceable promoters to activate zlmda24 expression in vivo are known in the art and can be used in this system to assess zlmda24 induction of metastasis. Moreover, purified zlmda24 or zlmda24-conditioned media can be directly injected in to this mouse model, and hence be used in this system. For general reference see, O'Reilly MS, et al. Cell 79:315-328, 1994; and Rusciano D, et al. Murine Models of Liver Metastasis. Invasion Metastasis 14:349-361, 1995.

Polynucleotides encoding zlmda24 polypeptides are useful within gene therapy applications where it is desired to increase or inhibit zlmda24 activity. If a mammal has a mutated or absent zlmda24 gene, the zlmda24 gene can be introduced into the cells of the mammal. In one embodiment, a gene encoding a zlmda24 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zlmda24 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Antisense methodology can be used to inhibit zlmda24 gene transcription, such as to inhibit cell proliferation in vivo. Polynucleotides that are complementary to a segment of a zlmda24-encoding polynucleotide (e.g., a polynucleotide as set froth in SEQ ID NO:1) are designed to bind to zlmda24-encoding mRNA and to inhibit translation of such mRNA. Such antisense polynucleotides are used to inhibit expression of zlmda24 polypeptide-encoding genes in cell culture or in a subject.

The present invention also provides reagents that will find use in diagnostic applications. For example, the zlmda24 gene, a probe comprising zlmda24 DNA or RNA or a subsequence thereof can be used to determine if the zlmda24 gene is present on a human chromosome, such as chromosome 7, or if a mutation has occurred. Based on annotation of a fragment of human genomic DNA containing a part of zlmda24 genomic DNA (Genbank Accession No. AC007458), zlmda24 is located at the 7q21 region of chromosome 7. Detectable chromosomal aberrations at the zlmda24 gene locus include, but are not limited to, aneuploidy, gene copy number changes, loss of heterogeneity (LOH), translocations, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

The precise knowledge of a gene's position can be useful for a number of purposes, including: 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have.

The zlmda24 gene is located at the 7q21 region of chromosome 7. Several genes of known function map to this region that are linked to human disease. For example, an increase in the copy number of chromosome 7 is the most common chromosomal abnormality observed in human malignant gliomas (Bigner, S. H. et al., Cancer Res. 48:405-411, 1998; Bigner, S. H. et al., J. Neuropathol. Exp. Neurol. 47:191-205, 1998; Bigner, S. H. et al., Atlas Sci. Biochem. 333-336, 1998). Thus, since the zlmda24 gene maps to chromosome 7, the zlmda24 polynucleotide probes of the present invention can be used to detect chromosome 7 trisomy and other chromosome 7 gains, and particularly chromosome 7q21 chromosome gain associated with human malignant gliomas. Hence, the polynucleotides of the present invention can serve as a diagnostic for human malignant gliomas. Moreover, trisomy of chromosome 7 is often found in papillary renal carcinoma tumors and the increase in copy number of mutated MET proto-oncogene (7q31) is believed to be a factor in tumorigenesis (Zhuang, Z et al., Nature Genet. 20:66-69, 1998). Thus, the zlmda24 polynucleotide probes of the present invention can be used to detect chromosome 7 trisomy associated with papillary renal carcinoma tumors. Hence, the polynucleotides of the present invention can serve as a diagnostic for papillary renal carcinoma tumors. Moreover, several chromosomal aberrations at 7q21 including deletions, rearrangements, and chromosomal breakpoints, and translocations are seen in humans with ectrodactyly, for instance seen in split hand/foot malformation (SHFM1). Moreover, the critical region for ectrodactyly is the 7q21.1-7q22.1 locus of chromosome 7. Thus, since the zlmda24 gene maps to this critical region, the zlmda24 polynucleotide probes of the present invention can be used to detect chromosome deletions, translocations and rearrangements associated with ectrodactyly. Moreover, chromosomal deletions of 7q21.1-q22 are associated with mucopolysaccharidosis type VII (MPS VII), which is manifested by mental retardation, amongst other phenotypic abnormalities. Similarly, zlmda24 polynucleotide probes of the present invention can be used to detect chromosome deletions, translocations and rearrangements associated with MPS VII. Moreover, the multi drug resistance 3 gene (MDR3) gene, mutations that confer drug resistance to hydrophobic drugs, e.g., chemotherapeutics, maps to 7q21.1. Moreover, amongst other genetic loci, those for Cholistasis (7q21.1), collagen abnormalities including maternal disomy (i.e., isodisomy) of chromosome 7 (7q22.1) and deletions (17q21-q22), frontonasal dysplasia (7q21), Zerwilliger syndrome (7q21-q22), and cerebral cavernous malformations (7q11-q21) all manifest themselves in human disease states as well as map to this region of the human genome. See the Online Mendellian Inheritance of Man (OMIM™, National Center for Biotechnology Information, National Library of Medicine. Bethesda, Md.) gene map, and references therein, for this region of human chromosome 7 on a publicly available world wide web server (http://www3.ncbi.nlm.nih.gov/htbin-post/Omim/getmap?chromosome=7q21). All of these serve as possible candidate genes for an inheritable disease that show linkage to the same chromosomal region as the zlmda24 gene. Thus, zlmda24 polynucleotide probes can be used to detect abnormalities or genotypes associated with these defects.

One of skill in the art would recognize that of zlmda24 polynucleotide probes are particularly useful for diagnosis of gross chromosomal abnormalities associated with loss of heterogeneity (LOH), chromosome gain (e.g. trisomy), translocation, DNA amplification, and the like. Translocations within chromosomal locus 7q21 wherein the zlmda24 gene is located are known to be associated with human disease. For example, 7q21 deletions and translocations, and trisomy are associated with malignant gliomas, papillary renal carcinoma tumors, and ectrodactyly as discussed above. Thus, since the zlmda24 gene maps to this critical region, zlmda24 polynucleotide probes of the present invention can be used to detect abnormalities or genotypes associated with 7q21 translocation, deletion and trisomy, and the like, described above.

Similarly, defects in the zlmda24 gene itself may result in a heritable human disease state. Molecules of the present invention, such as the polypeptides, antagonists, agonists, polynucleotides and antibodies of the present invention would aid in the detection, diagnosis prevention, and treatment associated with a zlmda24 genetic defect. As such, zlmda24 polynucleotides, polypeptides, and anti-zlmda24 antibodies serve an important use as a diagnostic to detect defects in the zlmda24 gene or protein, or defects in surrounding chromosomal regions at the 7q21 region of chromosome 7.

A diagnostic could assist physicians in determining the type of disease and appropriate associated therapy, or assistance in genetic counseling. As such, the inventive anti-zlmda24 antibodies, polynucleotides, and polypeptides can be used for the detection of zlmda24 polypeptide, mRNA or anti-zlmda24 antibodies, thus serving as markers and be directly used for detecting or genetic diseases or cancers, as described herein, using methods known in the art and described herein. Further, zlmda24 polynucleotide probes can be used to detect abnormalities or genotypes associated with chromosome 7q21 deletions and translocations associated with human diseases, such as those described above, or other translocations involved with malignant progression of tumors or other 7q21 mutations, which are expected to be involved in chromosome rearrangements in malignancy; or in other cancers.

As discussed above, defects in the zlmda24 gene itself may result in a heritable human disease state. Molecules of the present invention, such as the polypeptides, antagonists, agonists, polynucleotides and antibodies of the present invention would aid in the detection, diagnosis prevention, and treatment associated with a zlmda24 genetic defect. In addition, zlmda24 polynucleotide probes can be used to detect allelic differences between diseased or non-diseased individuals at the zlmda24 chromosomal locus. As such, the zlmda24 sequences can be used as diagnostics in forensic DNA profiling.

In general, the diagnostic methods used in genetic linkage analysis, to detect a genetic abnormality or aberration in a patient, are known in the art. Most diagnostic methods comprise the steps of (a) obtaining a genetic sample from a potentially diseased patient, diseased patient or potential non-diseased carrier of a recessive disease allele; (b) producing a first reaction product by incubating the genetic sample with a zlmda24 polynucleotide probe wherein the polynucleotide will hybridize to complementary polynucleotide sequence, such as in RFLP analysis or by incubating the genetic sample with sense and antisense primers in a PCR reaction under appropriate PCR reaction conditions; (iii) Visualizing the first reaction product by gel electrophoresis and/or other known method such as visualizing the first reaction product with a zlmda24 polynucleotide probe wherein the polynucleotide will hybridize to the complementary polynucleotide sequence of the first reaction; and (iv) comparing the visualized first reaction product to a second control reaction product of a genetic sample from wild type patient. A difference between the first reaction product and the control reaction product is indicative of a genetic abnormality in the diseased or potentially diseased patient, or the presence of a heterozygous recessive carrier phenotype for a non-diseased patient, or the presence of a genetic defect in a tumor from a diseased patient, or the presence of a genetic abnormality in a fetus or pre-implantation embryo. For example, a difference in restriction fragment pattern, length of PCR products, length of repetitive sequences at the zlmda24 genetic locus, and the like, are indicative of a genetic abnormality, genetic aberration, or allelic difference in comparison to the normal wild type control. Controls can be from unaffected family members, or unrelated individuals, depending on the test and availability of samples. Genetic samples for use within the present invention include genomic DNA, mRNA, and cDNA isolated form any tissue or other biological sample from a patient, such as but not limited to, blood, saliva, semen, embryonic cells, amniotic fluid, and the like. The polynucleotide probe or primer can be RNA or DNA, and will comprise a portion of SEQ ID NO:1, the complement of SEQ ID NO:1, or an RNA equivalent thereof. Such methods of showing genetic linkage analysis to human disease phenotypes are well known in the art. For reference to PCR based methods in diagnostics see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

Aberrations associated with the zlmda24 locus can be detected using nucleic acid molecules of the present invention by employing standard methods for direct mutation analysis, such as restriction fragment length polymorphism analysis, short tandem repeat analysis employing PCR techniques, amplification-refractory mutation system analysis, single-strand conformation polymorphism detection, RNase cleavage methods, denaturing gradient gel electrophoresis, fluorescence-assisted mismatch analysis, and other genetic analysis techniques known in the art (see, for example, Mathew (ed.), Protocols in Human Molecular Genetics (Humana Press, Inc. 1991), Marian, Chest 108:255 (1995), Coleman and Tsongalis, Molecular Diagnostics (Human Press, Inc. 1996), Elles (ed.) Molecular Diagnosis of Genetic Diseases (Humana Press, Inc. 1996), Landegren (ed.), Laboratory Protocols for Mutation Detection (Oxford University Press 1996), Birren et al. (eds.), Genome Analysis, Vol. 2: Detecting Genes (Cold Spring Harbor Laboratory Press 1998), Dracopoli et al. (eds.), Current Protocols in Human Genetics (John Wiley & Sons 1998), and Richards and Ward, “Molecular Diagnostic Testing,” in Principles of Molecular Medicine, pages 83-88 (Humana Press, Inc. 1998)). Direct analysis of an zlmda24 gene for a mutation can be performed using a subject's genomic DNA. Methods for amplifying genomic DNA, obtained for example from peripheral blood lymphocytes, are well-known to those of skill in the art (see, for example, Dracopoli et al. (eds.), Current Protocols in Human Genetics, at pages 7.1.6 to 7.1.7 (John Wiley & Sons 1998)).

Mice engineered to express the zlmda24 gene, referred to as “transgenic mice,” and mice that exhibit a complete absence of zlmda24 gene function, referred to as “knockout mice,” may also be generated (Snouwaert et al., Science 257:1083, 1992; Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express zlmda24, either ubiquitously or under a tissue-specific or tissue-restricted promoter can be used to ask whether over-expression causes a phenotype. For example, over-expression of a wild-type zlmda24 polypeptide, polypeptide fragment or a mutant thereof may alter normal cellular processes, resulting in a phenotype that identifies a tissue in which zlmda24 expression is functionally relevant and may indicate a therapeutic target for the zlmda24, its agonists or antagonists. For example, a preferred transgenic mouse to engineer is one that over-expresses the mature zlmda24 polypeptide (amino acid residues 32 (His) to 253 (Phe) of SEQ ID NO:2; or amino acid residues 28 (Thr) to 253 (Phe) of SEQ ID NO:4). Moreover, such over-expression may result in a phenotype that shows similarity with human diseases. Similarly, knockout zlmda24 mice can be used to determine where zlmda24 is absolutely required in vivo. The phenotype of knockout mice is predictive of the in vivo effects of that a zlmda24 antagonist, such as those described herein, may have. The human or mouse zlmda24 cDNA can be used to generate knockout mice. These transgenic or knockout mice may be employed to study the zlmda24 gene and the protein encoded thereby in an in vivo system, and can be used as in vivo models for corresponding human diseases. Moreover, transgenic mice expression of zlmda24 antisense polynucleotides or ribozymes directed against zlmda24, described herein, can be used analogously to transgenic mice described above. Studies may be carried out by administration of purified zlmda24 protein, as well.

The zlmda24 transgenic and knockout mice may be employed to study the zlmda24 gene and the protein encoded thereby in an in vivo system, and can be used as in vivo models for corresponding human or animal diseases (such as those in commercially viable animal populations). The mouse models of the present invention are particularly relevant as tumor models for the study of cancer biology and progression, testicular cancer and disease, fertility, and as models of cardiomyopathy and heart disease, ectopic thyroid disease, and hematopoietic and lymphoid diseases. Such models are useful in the development and efficacy of therapeutic molecules used in human disease and fertility. Because zlmda24 expression, are associated with specific human tissues and conditions, both transgenic mice and knockout mice would serve as useful animal models. Moreover, in a preferred embodiment, zlmda24 transgenic mouse can serve as an animal model for specific tumors, particularly testicular cancer. Moreover, transgenic mice expression of zlmda24 antisense polynucleotides or ribozymes directed against zlmda24, described herein, can be used analogously to transgenic mice described above.

Polynucleotides and polypeptides of the present invention will additionally find use as educational tools as a laboratory practicum kits for courses related to genetics and molecular biology, protein chemistry and antibody production and analysis. Due to its unique polynucleotide and polypeptide sequence molecules of zlmda24 can be used as standards or as “unknowns” for testing purposes. For example, zlmda24 polynucleotides can be used as an aid, such as, for example, to teach a student how to prepare expression constructs for bacterial, viral, and/or mammalian expression, including fusion constructs, wherein zlmda24 is the gene to be expressed; for determining the restriction endonuclease cleavage sites of the polynucleotides; determining mRNA and DNA localization of zlmda24 polynucleotides in tissues (i.e., by Northern and Southern blotting as well as polymerase chain reaction); and for identifying related polynucleotides and polypeptides by nucleic acid hybridization.

Zlmda24 polypeptides can be used educationally as an aid to teach preparation of antibodies; identifying proteins by Western blotting; protein purification; determining the weight of expressed zlmda24 polypeptides as a ratio to total protein expressed; identifying peptide cleavage sites; coupling amino and carboxyl terminal tags; amino acid sequence analysis, as well as, but not limited to monitoring biological activities of both the native and tagged protein (i.e., receptor binding, signal transduction, proliferation, and differentiation) in vitro and in vivo. Zlmda24 polypeptides can also be used to teach analytical skills such as mass spectrometry, circular dichroism to determine conformation, especially of the four alpha helices, x-ray crystallography to determine the three-dimensional structure in atomic detail, nuclear magnetic resonance spectroscopy to reveal the structure of proteins in solution. For example, a kit containing the zlmda24 can be given to the student to analyze. Since the amino acid sequence would be known by the professor, the specific protein can be given to the student as a test to determine the skills or develop the skills of the student, the teacher would then know whether or not the student has correctly analyzed the polypeptide. Since every polypeptide is unique, the educational utility of zlmda24 would be unique unto itself.

Moreover, since zlmda24 has a testis-specific expression and is a polypeptide with a four-helix bundle structure and a distinct chromosomal localization, activity can be measured using proliferation assays; luciferase and binding assays described herein. Moreover, expression of zlmda24 polynucleotides and polypeptides in testicular and other tissues can be analyzed in order to train students in the use of diagnostic and tissue-specific identification and methods. Moreover zlmda24 polynucleotides can be used to train students on the use of chromosomal detection and diagnostic methods, since it's locus is known. Moreover, students can be specifically trained and educated about human chromosome 7, and more specifically the 7p21 locus. Such assays are well known in the art, and can be used in an educational setting to teach students about alpha-helical proteins and examine different properties, such as cellular effects on cells, enzyme kinetics, varying antibody binding affinities, tissue specificity, and the like, between zlmda24 and other cytokine-like polypeptides in the art.

The antibodies which bind specifically to zlmda24 can be used as a teaching aid to instruct students how to prepare affinity chromatography columns to purify zlmda24, cloning and sequencing the polynucleotide that encodes an antibody and thus as a practicum for teaching a student how to design humanized antibodies. Moreover, antibodies which bind specifically to zlmda24 can be used as a teaching aid for use in detection of testicular tissue using histological, and in situ methods amongst others known in the art. The zlmda24 gene, polypeptide or antibody would then be packaged by reagent companies and sold to universities so that the students gain skill in art of molecular biology. Because each gene and protein is unique, each gene and protein creates unique challenges and learning experiences for students in a lab practicum. Such educational kits containing the zlmda24 gene, polypeptide or antibody are considered within the scope of the present invention.

For pharmaceutical use, the polypeptides or antibodies of the present invention are formulated for parenteral, particularly intravenous or subcutaneous, delivery according to conventional methods. Intravenous administration will be by bolus injection or infusion over a typical period of one to several hours. In general, pharmaceutical formulations will include a zlmda24 protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19th ed., 1995. Therapeutic doses will generally be in the range of 0.1 to 100 μg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years. In general, a therapeutically effective amount of zlmda24 is an amount sufficient to produce a clinically significant change in hematopoietic or immune function.

The invention is further illustrated by the following non-limiting examples.

### EXAMPLES

### Example 1

### Isolation and Cloning of Human and Mouse zlmda24

A. Obtaining Full-length Human zlmda24

Scanning of an in-house generated human cDNA database of sequenced cDNAs from testis library resulted in identification of part of the human zlmda24 sequence. Subsequently, an expressed sequence tag (EST) was identified. A plasmid clone was obtained and sequenced; a polynucleotide encoding a full-length human zlmda24 polypeptide was isolated. The cDNA sequence for human zlmda24 was full length, as shown in SEQ ID NO:1. The corresponding amino acid sequence for human zlmda24 is shown in SEQ ID NO:2.

B. Isolation of Full Length zlmda24 cDNA: Screening Mouse Testis cDNA Library

Scanning of a translated murine cDNA database against human zlmda24 resulted in identification of an expressed sequence tag (EST) sequence (EST3295730). A mouse probe, based on the identified EST was generated by PCR using oligos ZC29,714 (SEQ ID NO:7) and ZC29,984 (SEQ ID NO:8), and in-house mouse testis library as a template under the following reaction conditions: 94° C. for 5 minutes; 35 cycles of 94° C. for 30 seconds, 65° C. for 30 seconds, 72° C. for 30 second followed by 72° C. for 7 minutes. The PCR fragment was gel purified using QIAquick gel extraction kit (Qiagen).

The mouse testis library was an arrayed library representing 9.6×10⁵ clones. The library was screened using the PCR conditions described above. The library was deconvoluted down to a positive pool of 250 colonies. The positive mouse testis pool was plated and filter-lifted using Hybond-N filters (Amersham, England). A total of about 1000 colonies were screened on 4 filters lifted from plates of about 250 colonies per plate. The filters were marked with a hot needle for orientation, then denatured for 6 minutes in 0.5 M NaOH and 1.5 M Tris-HCL pH 7.2. The filters were then neutralized in 1.5 M NaCl and 0.5 M Tris-HCL pH 7.2 for 6 minutes. The DNA was affixed to the filters using a Stratalinker UV crosslinker (Stratagene, La Jolla, Calif.) at 1200 joules. The filters were prewashed at 65 degrees C. in prewash buffer consisting of 0.25×SSC, 0.25% SDS and 1 mM EDTA. The solution was changed a total of three times over a 45-minute period to remove cell debris. Filters were prehybridized overnight at 65° C. in 25ml Expresshyb (Clontech, Palo Alto, Calif.).

The probe was generated by PCR using oligos ZC29714 (SEQ ID NO:7) and ZC29984 (SEQ ID NO:8), as described above. The PCR fragment was gel purified using QIAquick gel extraction kit (Qiagen, Santa Clarita, Calif.). The probe was radioactively labeled with ³²p using the Rediprime II DNA Labeling system (Amersham, UK) according to Manufacturer's specifications. The probe was purified using a Nuctrap push column (Stratagene cloning system, La Jolla, Calif.). Expresshyb (Clontech, Palo Alto, Calif.) solution was used for the hybridizing solution for the filters. Hybridization took place overnight at 65° C. Filters were rinsed 2× in 65° C. in pre-wash buffer (0.25×SSC, 0.25% SDS and 1mM EDTA). Then the filters were washed 2× in pre-wash solution at 65° C. Filters were exposed to film for 1 days at −80° C.

There was 6 positive clones on the filters. These 6 clones were picked and streaked out to obtain individual colonies. 11 individual colonies were subsequently screened by PCR, using oligos ZC29,714 (SEQ ID NO:7) and ZC29,984 (SEQ ID NO:8) as described above. Two clones were sequenced to verify the sequence. The cDNA sequence for mouse zlmda24 was full length and double stranded, and is shown in SEQ ID NO:3. The corresponding amino acid sequence for mouse zlmda24 is shown in SEQ ID NO:4.

### Example 2

### Human Zlmda24 Tissue Distribution Using PCR

A panel of cDNAs from human tissues was screened for zlmda24 expression using PCR. The panel was made in-house and contained 94 marathon cDNA and cDNA samples from various normal and cancerous human tissues and cell lines and is shown in Table 5, below. The cDNAs came from in-house libraries or marathon cDNAs from in-house RNA preps, Clontech RNA, or Invitrogen RNA. The marathon cDNAs were made using the marathon-Ready™ kit (Clontech, Palo Alto, Calif.) and QC tested with clathrin primers ZC21195 (SEQ ID NO:9) and ZC21196 (SEQ ID NO:10) and then diluted based on the intensity of the clathrin band. To assure quality of the panel samples, three tests for quality control (QC) were run: (1) To assess the RNA quality used for the libraries, the in-house cDNAs were tested for average insert size by PCR with vector oligos that were specific for the vector sequences for an individual cDNA library; (2) Standardization of the concentration of the cDNA in panel samples was achieved using standard PCR methods to amplify full length alpha tubulin or G3PDH cDNA using a 5′ vector oligo ZC14,063 (SEQ ID NO:11) and 3′ alpha tubulin specific oligo primer ZC17,574 (SEQ ID NO:12) or 3′ G3PDH specific oligo primer ZC17,600 (SEQ ID NO:13); and (3) a sample was sent to sequencing to check for possible ribosomal or mitochondrial DNA contamination. The panel was set up in a 96-well format that included a human genomic DNA (Clontech, Palo Alto, Calif.) positive control sample. Each well contained approximately 0.2-100 pg/μl of cDNA. The PCR reactions were set up using oligos ZC28587 (SEQ ID NO:14) and ZC28591 (SEQ ID NO:15), Advantage Taq™ (Clontech), and Rediload dye (Research Genetics, Inc., Huntsville, Ala.). The amplification was carried out as follows: 1 cycle at 94° C. for 2 minutes, 35 cycles of 94° C. for 30 seconds, 60.0° C. for 30 seconds and 72° C. for seconds, followed by 1 cycle at 72° C. for 10 minutes. About 10 μl of the PCR reaction product was subjected to standard Agarose gel electrophoresis using a 4% agarose gel. The correct predicted DNA fragment size was primarily observed in testis (i.e., all the testis samples on plate), and very weakly in salivary gland. The other tissues and cell lines tested were negative, indicating that the expression of zlmda24 is testis-specific.

TABLE 5

| | # | | # |
|---|---|---|---|
| Tissue/Cell line | samples | Tissue/Cell line | samples |
| Adrenal gland | 1 | Bone marrow | 3 |
| Bladder | 1 | Fetal brain | 3 |
| Bone Marrow | 1 | Islet | 2 |
| Brain | 1 | Prostate | 3 |
| Cervix | 1 | RPMI #1788 | 2 |
| | | (ATCC # CCL-156) | |
| Colon | 1 | Testis | 4 |
| Fetal brain | 1 | Thyroid | 2 |
| Fetal heart | 1 | WI38 (ATCC # CCL-75 | 2 |
| Fetal kidney | 1 | ARIP (ATCC # CRL-1674 - rat) | 1 |
| Fetal liver | 1 | HaCat - human keratinocytes | 1 |
| Fetal lung | 1 | HPV (ATCC # CRL-2221) | 1 |
| Fetal muscle | 1 | Adrenal gland | 1 |
| Fetal skin | 1 | Prostate SM | 2 |
| Heart | 2 | CD3+ selected PBMC's | 1 |
| | | Ionomycin + PMA stimulated | |
| K562 | 1 | HPVS (ATCC # CRL-2221)- | 1 |
| (ATCC # CCL-243) | | selected | |
| Kidney | 1 | Heart | 1 |
| Liver | 1 | Pituitary | 1 |
| Lung | 1 | Placenta | 2 |
| Lymph node | 1 | Salivary gland | 1 |
| Melanoma | 1 | HL60 (ATCC # CCL-240) | 3 |
| Pancreas | 1 | Platelet | 1 |
| Pituitary | 1 | HBL-100 | 1 |
| Placenta | 1 | Renal mesangial | 1 |
| Prostate | 1 | T-cell | 1 |
| Rectum | 1 | Neutrophil | 1 |
| Salivary Gland | 1 | MPC | 1 |
| Skeletal muscle | 1 | Hut-102 (ATCC # TIB-162) | 1 |
| Small intestine | 1 | Endothelial | 1 |
| Spinal cord | 1 | HepG2 (ATCC # HB-8065) | 1 |
| Spleen | 1 | Fibroblast | 1 |
| Stomach | 1 | E. Histo | 1 |
| Testis | 2 | | |
| Thymus | 1 | | |
| Thyroid | 1 | | |
| Trachea | 1 | | |
| Uterus | 1 | | |
| Esophagus tumor | 1 | | |
| Gastric tumor | 1 | | |
| Kidney tumor | 1 | | |
| Liver tumor | 1 | | |
| Lung tumor | 1 | | |
| Ovarian tumor | 1 | | |
| Rectal tumor | 1 | | |
| Uterus tumor | 1 | | |

### Example 3

### Chromosomal Assignment and Placement of Human Zlmda24

Zlmda24 was mapped to chromosome 7 using the commercially available “GeneBridge 4 Radiation Hybrid (RH) Mapping Panel” (Research Genetics, Inc., Huntsville, Ala.). The GeneBridge 4 RH panel contains DNA from each of 93 radiation hybrid clones, plus two control DNAs (the HFL donor and the A23 recipient). A publicly available WWW server (http://www-genome.wi.mit.edu/cgi-bin/contig/rhmapper.pl) allows mapping relative to the Whitehead Institute/MIT Center for Genome Research's radiation hybrid map of the human genome (the “WICGR” radiation hybrid map) which was constructed with the GeneBridge 4 RH panel.

For the mapping of Zlmda24 with the GeneBridge 4 RH panel, 20 μl reactions were set up in a 96-well microtiter plate compatible for PCR (Stratagene, La Jolla, Calif.) and used in a “RoboCycler Gradient 96” thermal cycler (Stratagene). Each of the 95 PCR reactions consisted of 2 μl 10×PCR reaction buffer (Qiagen, Inc., Valencia, Calif.), 1.6 μl dNTPs mix (2.5 mM each, PERKIN-ELMER, Foster City, Calif.), 1 μl sense primer, ZC 29,567 (SEQ ID NO:16), 1 μl antisense primer, ZC 29,568 (SEQ ID NO:17), 2 μl “RediLoad” (Research Genetics, Inc., Huntsville, Ala.), 0.1 μl Qiagen HotStarTaq DNA Polymerase (5 units/μl), 25 ng of DNA from an individual hybrid clone or control and distilled water for a total volume of 20 μl. The reactions were overlaid with an equal amount of mineral oil and sealed. The PCR cycler conditions were as follows: an initial 1 cycle 15 minute denaturation at 95° C., 35 cycles of a 1 minute denaturation at 95° C., 1 minute annealing at 48° C. and 1 minute and 15 seconds extension at 72° C., followed by a final 1 cycle extension of 7 minutes at 72° C. The reactions were separated by electrophoresis on a 2% agarose gel (EM Science, Gibbstown, N.J.) and visualized by staining with ethidium bromide.

The results showed that Zlmda24 maps 5.45 cR_{—}3000 distal from the framework marker D7S644 on the chromosome 7 WICGR radiation hybrid map. The use of surrounding genes/markers positions Zlmda24 in the 7q21 chromosomal region.

### Example 4

### Baculovirus Expression of Zlmda24CEE

An expression vector, pzlmda24CEE, was prepared to express zlmda24 polypeptides in insect cells. PzlmbdaCEE, was designed to express a zlmda24 polypeptide with a C-terminal GLU-GLU tag (SEQ ID NO:18). This construct can be used to determine the N-terminal amino acid sequence of zlmda24 after the signal peptide has been cleaved off.

A. Construction of Pzlmbda24CEE

A zlmda24 fragment containing BamHI and XbaI restriction sites on the 5′ and 3′ ends, respectively, was generated by PCR amplification from a plasmid containing zlmda24 cDNA (Example 1) using primers ZC29,055 (SEQ ID NO:19) and ZC29,056 (SEQ ID NO:20). Due to the method of cloning, there are two introduced amino acids (Ala-Leu) prior to the Glu-Glu tag (SEQ ID NO:18) that come from the Xba cloning site. The PCR reaction conditions were as follows: 20 cycles of 94° C. for 30 seconds, 77° C. for 60 seconds; 1 cycle at 72° C. for 7 min; followed by 4° C. soak. The fragment was visualized by gel electrophoresis (1% SeaPlaque/1% NuSieve). The band was excised and purified using a Qiagen Gel Extraction Kit according to the manufacturer's instructions. The purified fragment was then subcloned into a PCR 2.1 TOPO vector (Invitrogen) according to the manufacturer's instructions. A subclone with the correct zlmda24 sequence was identified by sequencing. Plasmid DNA from the correct subclone was digested with BamHI and Xba I and ligated into an BamHI/XbaI digested baculovirus expression vector, pZBV32L. The pZBV32L vector is a modification of the pFastBac 1™ (Life Technologies) expression vector, where the polyhedron promoter has been removed and replaced with the late activating Basic Protein Promoter, and the coding sequence for the Glu-Glu tag as well as a stop signal is inserted at the 3′ end of the multiple cloning region). About 25 nanograms of the restriction digested zlmda24 insert and about 32 ng of the corresponding vector were ligated overnight at 16° C. The ligation mix was diluted 3 fold in TE (10 mM Tris-HCl, pH 7.5 and 1 mM EDTA) and 4 fmol of the diluted ligation mix was transformed into DH10b electrocompetent cells (Life Technologies) according to manufacturer's direction. The transformed DNA and cells were diluted in 450 μl of SOC media (2% Bacto Tryptone, 0.5% Bacto Yeast Extract, 10 ml 1M NaCl, 1.5 mM KCl, 10 mM MgCl₂, 10 mM MgSO₄ and 20 mM glucose) and plated onto LB plates containing 100 μg/ml ampicillin. Clones were analyzed by restriction digests. The zlmda24-CEE polypeptide is shown in SEQ ID NO:23.

### Example 5

### Baculovirus Expression of Zlmda24-CEE

An expression vector, zlmda24-CEE/pZBV32L, was prepared to express zlmda24-CEE polypeptides in insect cells. zlmda24-CEE/pZBV32L was designed to express a zlmda24 polypeptide with a C-terminal GLU-GLU tag (SEQ ID NO:23) (Example 3 and Example 4). This construct can be used to determine the N-terminal amino acid sequence of zlmda24 after the signal peptide has been cleaved off.

### A. Viral Generation of zlmda24-CEE/pZBV32L

Two μl of the expression vector zlmda24-CEE/pZBV32L was transformed into 20 μl DH10Bac™ Max Efficiency® competent cells (GIBCO-BRL) by heat shock for 45 seconds in a 42° C. heat block. The transformed DH10Bac™ cells were diluted in 980 μl SOC media (2% Bacto Tryptone, 0.5% Bacto Yeast Extract, 10 ml 1M NaCl, 1.5 mM KCl, 10 mM MgCl₂, 10 mM MgSO₄ and 20 mM glucose) and 100 μl were plated onto Luria Agar plates containing 50 μg/ml kanamycin, 7 μg/ml gentamicin, 10 μg/ml tetracycline, 40 μg/ml IPTG and 200 μg/ml Bluo Gal. The plates were incubated for 48 hours at 37° C. A color selection was used to identify those cells having transposed viral DNA (referred to as a “bacmid”). Those colonies, which were white in color, were picked for analysis. Colonies were analyzed by PCR and positive colonies (containing desired bacmid) were selected for outgrowth and purified using a QLAprep® Spin Miniprep Kit (Qiagen). Clones were screened for the correct insert by amplifying DNA using primers to the transposable element in the bacmid via PCR using primers ZC447 (SEQ ID NO:21) and ZC976 (SEQ ID NO:22). The PCR reaction conditions were as follows: 1 cycle at 94° C. for 5 minutes; 30 cycles of 94° C. for 60 seconds, 50° C. for 90 seconds, and 72° C. for 180 seconds; 1 cycle at 72° C. for 10 m followed by 4° C. soak. The PCR product was run on a 1% agarose gel to check the insert size. Those having the correct insert were used to transfect Spodoptera Frugiperda (Sf9) cells.

B. Transfection

Sf9 cells were seeded at 1×10⁶ cells per well in a 6-well plate and allowed to attach for 1 hour at 27° C. Five microliters of bacmid DNA were diluted with 100 μl Sf-900 II SFM (Life Technologies). Twenty μl of Lipofectamine™ Reagent (Life Technologies) were diluted with 100 μl Sf-900 II SFM. The bacmid DNA and lipid solutions were gently mixed and incubated 30-45 minutes at room temperature. The media from one well of cells was aspirated, the cells were washed 1× with 2 ml fresh Sf-900 II SFM media. Eight hundred microliters of Sf-900 II SFM was added to the lipid-DNA mixture. The wash media was aspirated and the DNA-lipid mix added to the cells. The cells were incubated at 27° C. overnight. The DNA-lipid mix was aspirated and 2 ml of Sf-900 II media was added to each plate. The plates were incubated at 27° C., 90% humidity, for 96 hours after which the virus was harvested.

C. Amplification

Sf9 cells were seeded at 1×10⁶ cells per well in a 6-well plate. 50 μl of virus from the transfection plate were placed in the well and the plate was incubated at 27° C., 90% humidity, for 96 hours after which the virus was harvested.

Sf9 cells were grown in 50 ml Sf-900 II SFM in a 125 ml shake flask to an approximate density of 1×10⁶ cells/ml. They were then infected with 100 μl of the viral stock from the above plate and incubated at 27° C. for 3 days after which time the virus was harvested.

### Example 6

### Purification of Zlmda24-CEE from Sf9 Cells

The following procedure was used to purify zlmda24 polypeptides containing C-terminal Glu-Glu (EE) tags (SEQ ID NO:23). 50 ml of conditioned media from Sf9 cells at 2×10⁶ cells/ml expressing zlmda24-CEE (Example 5) was filtered using a 0.22 μm Steriflip™ filter (Millipore) and one Complete™ protease inhibitor cocktail tablet (Boerhinger) was added for every 50 ml of media. The cell pellet from the 50 ml expression culture was harvested and 10 ml of cell lysate buffer (150 mM Sodium Chloride, 50 mM Tris pH 8.0, and 1% NP-40) was added along with one Complete™ protease inhibitor cocktail tablet (Boerhinger). Total target protein concentrations of the conditioned media and cell lysate were determined via standard SDS-PAGE and Western blot analysis using an anti-EE antibody followed by a secondary anti-mlg HRP conjugated antibody. Results indicated that the protein was not secreted into the media, but was associated with the cells.

Batch purification was accomplished by adding 250 μl of Protein G Sepharose® 4 Fast Flow (Pharmacia) which was treated with anti-EE antibody, to 40 ml of Sf9 conditioned media and 10 ml of cell lysate. The media/lysate-bead mixture was rocked overnight at 4° C. The beads were spun out of the media at 1000 RPM for 10 minutes in a Beckman GS6R centrifuge. The beads were washed using the following scheme (centrifugation and aspiration steps were done after each wash): 1× with 1 ml cell lysis buffer (150 mM Sodium Chloride, 50 mM Tris pH 8.0, and 1% NP-40); 1× with 1 ml wash buffer (650 mM Sodium Chloride, 50 mM Tris pH 8.0, and 1% NP-40); 1× with 1 ml cell lysis buffer. The beads were then suspended in 500 μl cell lysis buffer and submitted for N-terminal sequencing.

### Example 7

### Construct for Generating Human Zlmda24 Transgenic Mice

Oligonucleotides were designed to generate a PCR fragment containing a consensus Kozak sequence and the exact human zlmda24 coding region. These oligonucleotides were designed with an FseI site at the 5′ end and an AscI site at the 3′ end to facilitate cloning into the pTg12-8 MT expression vector.

PCR reactions were carried out using Advantage® cDNA polymerase (Clontech) to amplify a human zlmda24 cDNA fragment. About 200 ng of human zlmda24 polynucleotide template (Example 1), and oligonucleotides ZC29127 (SEQ ID NO:24) and ZC29128 (SEQ ID NO:25) were used in the PCR reaction. PCR reaction conditions were as follows: 95° C. for 5 minutes; 15 cycles of 95° C. for 60 seconds, 60° C. for 60 seconds, and 72° C. for 90 seconds; and 72° C. for 7 minutes; followed by a 4° C. hold. PCR products were separated by agarose gel electrophoresis and purified using a QiaQuick™ (Qiagen) gel extraction kit. The isolated, approximately 761 bp, DNA fragment was digested with FseI and AscI (New England BioLabs), ethanol precipitated and ligated into pTg12-8 MT that was previously digested with FseI and AscI. The pTg12-8 MT plasmid, designed for expression of a gene of interest in transgenic mice, contains an expression cassette flanked by 10 kb of MT-1 5′ DNA and 7 kb of MT-1 3′ DNA. The expression cassette is comprised of the MT-1 promoter, the rat insulin II intron, a polylinker for the insertion of the desired clone, and the human growth hormone poly A sequence.

About one microliter of the ligation reaction was electroporated into DH10B ElectroMax® competent cells (GIBCO BRL, Gaithersburg, Md.) according to manufacturer's direction and plated onto LB plates containing 100 μg/ml ampicillin, and incubated overnight. Colonies were picked and grown in LB media containing 100 μg/ml ampicillin. Miniprep DNA was prepared from the picked clones and screened for the zlmda24 insert by restriction digestion with EcoRI and subsequent agarose gel electrophoresis and analysis. Maxipreps of the correct pTg12-8 MT zlmda24 construct, as verified by sequence analysis, were performed. A SalI fragment containing the 5′ and 3′ flanking sequences, the MT promoter, the rat insulin II intron, zlmda24 cDNA and the human growth hormone poly A sequence was prepared and used for microinjection into fertilized murine oocytes.

### Example 8

### Zlmda24 Transgenic Mice

A. Generation of Transgenic Mice Expressing Human and Mouse Zlmda24

DNA fragments from transgenic vectors (Example 7) containing 5′ and 3′ flanking sequences of the MT-1 promoter, the rat insulin II intron, zlmda24 cDNA and the human growth hormone poly A sequence were prepared and used for microinjection into fertilized B6C3f1 (Taconic, Germantown, N.Y.) murine oocytes, using a standard microinjection protocol. See, Hogan, B. et al., Manipulating the Mouse Embryo. A Laboratory Manual, Cold Spring Harbor Laboratory Press, 1994.

Ten transgenic mice expressing human zlmda24 from the MT-1 promoter were identified among 54 pups. Four pups died one day after birth. Expression levels in the weaned mice was analyzed, with 3 mice exhibiting high expression, 5 mice exhibiting medium expression, and 2 mice exhibiting low expression. Tissues were prepared and histologically examined as describe below.

B. Microscopic Evaluation of Tissues from Transgenic Mice

Histopathological observation of one pup found dead (day after birth) and littermate control (LMC), note that the tissues were immature, consistent with their young age. Distention of the atria of the heart was considered to be a feature of immaturity.

Male and female transgenic animals and LMC were examined at 8 weeks of age. Among the five females, one heart was found to have cardiomyopathy, and 1 of had ectopic thymus. Among the 4 necropsied males, 1 kidney was found with infiltrated mononuclear cells, and 1 with ectopic thymus.

No significant changes were observed in body weight, tissue weights, nor blood parameters, but the transgenic brains and thymuses tended to cluster in the lighter range, their calcium levels clustered in the lower range, and the total bilirubin in the higher range for this experiment.

The cardiomyopathy, ectopic thymus, and kidney infiltration could signify a role for zlmda24 in autoimmunity and inflammation. Separately, the ectopic thymus might indicate that the zlmda24 cytokine has lymphopoeitic activity. Such lymphopoeitic activity can be useful for cancer, virus infection, and immune reconstitution in a broad range of immunosuppressed patients, immunotherapy, as discussed herein.

One transgenic female was found to have cardiomyopathy which literally means “disease of heart muscle”, and includes several distinct conditions, including Arrhythmogenic Right Ventricular Cardiomyopathy (ARVC), Dilated Cardiomyopathy (DCM), Hypertrophic Cardiomyopathy (HCM), and Restrictive Cardiomyopathy (RCM). Since MT-1-directed expression may have caused this pathology, zlmda24 could play a role in initiation or maintenance of such human disease states. Thus, a zlmda24 antagonist, such as a zlmda24 polypeptide variant, an anti-zlmda24 antibody, binding peptide, or the like could be used to treat these, or other hypertrophic conditions of the heart muscle. This indication is explored by additional transgenic experiments, including expression of zlmda24 in cardiac myocytes, using a cardiac-myosin specific promoter, or by additional systemic expression promoters. Expression of zlmda24 driven by a lymphoid-specific promoter, such as EuLCK, can also show this effect, if mediated through elements of the innate or adaptive immune responses. Moreover, transgenics expressing zlmda24 from a lymphoid-specific promoter can be used to assess effects on mature lymphocyte populations and their functions. Alternatively, these effects are confirmed by injection of purified zlmda24 protein.

Additional CBC data representing circulating levels of mature blood cell components is used to determine whether or not the zlmda24 has an effect on hematopoiesis, despite its apparent tissue restriction to testis expression. Some proteins involved with hematopoiesis can have highly selective tissue expression and exhibit their effects elsewhere in the body, and particularly in lymphoid and hematopoietic cells. For example, the WSX-2 receptor (IL-13Ra2) has testis-specific expression, but is a natural antagonist of IL-13, whose primary biologic role is in regulation of the TH2 immune responses and has no apparent significant function in the testis (Donaldson, D. D., et al., J. Immunology 161:2317-2324 (1998); Caput, D., et al., JBC 271: 16921 (1996)). In vitro assays of mature lymphocyte function, as well as in bone-marrow assays of hematopoietic function are described herein, known in the art, and can be used to test for such function of zlmda24.

### Example 9

### Expression of Zlmda24 in Cancer and Normal Tissues and Cell Lines Using Microarray Gene Expression Information

A. The Microarray Approach to mRNA Expression Analysis

Gene expression information for zlmda24 was obtained from oligonucleotide and/or cDNA microarrays. Microarrays show the mRNA expression level of a large number of genes across a large number of cell types or cells exposed to various conditions, or cells in various replication steps, depending on the experiment. Because all of the information for all of the genes on any given microarray is obtained from the same biological experiment, and all biological experiments employing the same microarray provide results on the same set of genes, it is possible to examine not only the expression pattern of a given gene in various tissues, cell lines, cancers, growth conditions, or cell cycle points, but also to compare the expression patterns between different genes.

Briefly, microarray experiments are conducted by extracting the mRNA from various tissues(s) or cell line(s) and analyzing it in ‘2-color’ or ‘single-color’ experiments. In a 2-color experiment, the mRNAs are reverse transcribed to cDNA in a reaction along with a fluorescent dye. Typically, one of the cDNA samples serves as a ‘reference standard’ and is labeled with one of the dyes. In separate reactions, mRNA from the ‘experimental’ tissue(s) or cell line(s) are likewise reverse transcribed to cDNA but in the presence of a dye label with a different emission wavelength from the reference. The cDNA experimental samples are then mixed with the reference sample and hybridized to the microarray. The microarray itself has thousands of unlabeled cDNA clones, PCR products, or oligonucleotides covalently bound as spots (also called ‘features’) on its surface. The labeled cDNAs thereby hybridize to their respective microarray spots. If a particular gene is transcribed at a higher level in the experimental sample relative to the reference, then the spot will fluoresce to a greater degree in the experimental sample dye wavelength channel. Conversely, if the gene in the experimental sample is down regulated, then the wavelength channel of the reference dye will be stronger. Finally, the microarrays are scanned at the wavelengths of both dyes and the results for each spot are recorded and stored electronically. Large numbers of microarray experiments are typically done together using the same reference cDNA, but with different experimental cDNAs obtained from various conditions, cell lines, tissues, time points, and the like. A single-color experiment is conducted similarly, except that a chromogenic or radioactive label is employed instead of a fluorescent dye. Also, in the ‘single-color’ experiment, the cDNAs are not mixed prior to hybridization, rather expression levels of the various genes are determined by the absolute intensity of the individual features on the microarray, and the data from ‘experimental’ samples are compared electronically with the data from the ‘reference’.

Raw and/or processed microarray expression information was obtained from a subscription and/or data that were electronically downloaded. Publicly available, purchased, or in-house custom designed software was used to analyze the microarray data. Prior to analysis, spots were examined to exclude experimental artifacts (dust spots, substrate imperfections, incomplete or uneven hybridization washes, etc.) and absorbance was adjusted to take into account background fluorescence of the microarray substrate. Very weak and very strong signals beyond the linear range response of the microarray reader were likewise excluded from analysis. In the case of 2-color experiments, analyses were typically done on the ratio of the absorbance intensities of the reference and sample wavelength channels for each spot. These absorbance ratios were normalized to log base 2. In the case of single-color experiments, the signal intensities for all spots in a given experiment were normalized to an average value of 0 with a standard deviation of 1.

Two types of information can be drawn from microarray experiments. At the simplest level, microarray results reveal the level of expression for a given gene under various conditions, analogous to a series of Northern experiments. At a higher level, one can examine the data sets to find genes that have a similar (or opposite) patterns of expression with a particular gene of interest. These methods of primary data analysis, and correlation data analysis are described below.

At a basic level, in the case of 2-color experiments, the ratio of fluorescence of the reference and sample wavelengths is a measure of the level of induction or repression of the experimental mRNA relative to the control (Ratio=[sample fluorescence/control reference fluorescence]). If there is no change in mRNA expression level of a given gene in the control and experimental samples, then the ratio for the corresponding spot will be 1. If the expression of the gene is induced in the experimental sample then the ratio of fluorescence for that spot will be greater than 1; if it is repressed then the ratio will be less than 1. In the case of single-color experiments, the fluctuation of the signal relative to the mean (0) is used as a measure of gene induction or repression. These fluctuations can be interpreted in a fashion similar to those of the 2-color experiments. Thus values less than 1 indicate repression, and values greater than 1 indicate induction. Table 6 and 7 show the level of expression of zlmda24 under a number of different conditions.

At a more complex level, the similarity of expression patterns between genes can be compared. Thus, the biological function of a gene may be elucidated by identifying genes with known functions and with similar or opposite expression profiles. One typical measure of the correlation of patterns is the Pearson's correlation (R). The Pearson correlation is a number between −1 and 1. Pairs of genes with highly correlated expression patterns will have a Pearson's correlation near 1. Those with no correlation to each other will be near 0. Pairs of genes for which the expression patterns are anti-correlated will be near −1. Although values between 0 and 1, or 0 and −1 can be considered positively or negatively correlated respectively, in the current analysis, correlations greater than 0.6 or less than −0.6 were considered to be significant. Thus genes related regulation or expression biology potentially related to zlmda24 were identified. Table 7 show the GenBank accession number (or clone id), description, and Pearson's correlation of genes which have expression patterns moderately to strongly correlated or anti-correlated with the expression pattern of zlmda24.

Oligonucleotide features corresponding to zlmda24 were found on the ‘Transcription_{—}35’ (or TXN35) microarray experiments (Cho, R J et al. Nat. Genet. 27:48-54, 2001). In these experiments Cho et al. employed a single-color approach to examine the expression patterns of primary fibroblasts over the course of 24 hours in the cell cycle. The cells were initially synchronized using a double thymidine-block protocol. These experiments were designed to identify novel genes involved in cell cycling and provide information on the function of previously uncharacterized transcripts. The Transcription_{—}35 data set is from experiments employing Affymetrix 35,000 human gene oligonucleotide arrays (Affymetrix, Inc., Santa Clara, Calif.). These experiments were conducted only one time for each (2 hour) time point. See, Cho et al. supra. for details of all these experiments. The resulting data were obtained from the public Web site through The Salk Institute, La Jolla, Calif. microarray chip data (http://www.salk.edu/docs/labs/chipdata/primary.html).

B. Determination of Zlmda24 Expression in Various Cell Lines, Tissue Types, and/or Growth Cycle Points in the TXN35 Experiment

Table 6 shows the expression profile of zlmda24 in the TXN35 experiment. Interestingly, zlmda24 was found to be upregulated in G1 arrest, as well as after 8 hours. Conversely, zlmda24 expression was relatively repressed at 2 and 6 hours post-arrest.

These results show that increase or decrease in expression of zlmda24 is correlated with cell cycle states. As such, detection of zlmda24 expression increase or decrease can be used as a diagnostic for certain cell cycle states or as a marker for certain cell cycle states. Use of polynucleotides, polypeptides, and antibodies of the present invention for such diagnostic purposes are known in the art, and disclosed herein.

TABLE 6
Expression profile of zlmda24 in the TXN35 microarray experiment.

| Experiment | Description | Expression |
|---|---|---|
| 0 hr | foreskin primary fibroblasts in G1 arrest, | 1.1 |
| | 35K feature, single experiment | |
| 2 hr | foreskin primary fibroblasts 2 hours post | −1.5 |
| | release from arrest, 35K feature, single | |
| | experiment | |
| 4 hr | foreskin primary fibroblasts 4 hours post | −0.5 |
| | release from arrest, 35K feature, single | |
| | experiment | |
| 6 hr | foreskin primary fibroblasts 6 hours post | −1.7 |
| | release from arrest, 35K feature, single | |
| | experiment | |
| 8 hr | foreskin primary fibroblasts 8 hours post | 1.3 |
| | release from arrest, 35K feature, single | |
| | experiment | |
| 10 hr | foreskin primary fibroblasts 10 hours post | 0.7 |
| | release from arrest, 35K feature, single | |
| | experiment | |
| 14 hr | foreskin primary fibroblasts 14 hours post | 0.7 |
| | release from arrest, 35K feature, single | |
| | experiment | |
| 16 hr | foreskin primary fibroblasts 16 hours post | 0.8 |
| | release from arrest, 35K feature, single | |
| | experiment | |
| 18 hr | foreskin primary fibroblasts 18 hours post | −1.3 |
| | release from arrest, 35K feature, single | |
| | experiment | |
| 20 hr | foreskin primary fibroblasts 20 hours post | 0.1 |
| | release from arrest, 35K feature, single | |
| | experiment | |
| 22 hr | foreskin primary fibroblasts 22 hours post | 0.4 |
| | release from arrest, 35K feature, single | |
| | experiment | |
| 24 hr | foreskin primary fibroblasts 24 hours post | 0.2 |
| | release from arrest, 35K feature, single | |
| | experiment | |

C. Determination of Genes Having Correlated and/or Anti-correlated Expression with Zlmda24 in the TXN35 Experiment

Table 7 shows the analysis of features in the TXN35 microarray that have a Pearson's correlation of expression greater than 0.6 and/or less than −0.6 with that of zlmda24. Features are indexed by the accession number of their corresponding gene or clone identifier, and the corresponding protein, if known, is described.

Our analysis of the data showed that zlmda24 had correlated expression (Pearson's R<−0.6 or >0.6) with 77 other features (Table 7). Several genes had a similar expression pattern to zlmda24 (See, table 7). In contrast, zlmda24 had an opposite expression profile with other genes (See, table 7). These results show that zlmda24 has an expression profile similar to the expression pattern of several genes involved in molecular, cellular, or biological processes.

TABLE 7
Genes with similar expression profiles to zlmda24 in the
TXN35 microarray experiment.

| | | R-value | |
|---|---|---|---|
| | | Description | |
| | | (or closest | |
| Accession | Locus | homolog) | Protein/Gene |
| AA243393 | EST882782 | 0.91 | Homo sapiens STE20- |
| | | | like kinase 3 (mst-3) |
| | | | mRNA, complete cds. |
| AA090295 | EST729143 | 0.88 | unknown |
| AA442830 | EST1087134 | 0.88 | unknown |
| Z38810 | EST63876 | 0.87 | Homo sapiens IMAGE |
| | | | Consortium ID 342479, |
| | | | mRNA sequence. |
| L13435 | HUMAGCGC | 0.86 | Human chromosome |
| | | | 3p21.2 gene sequence. |
| D82277 | EST446176 | 0.86 | Homo sapiens LDL |
| | | | induced EC protein |
| | | | mRNA, complete cds. |
| AA256990 | EST896514 | 0.85 | Homo sapiens PRO2751 |
| | | | mRNA, complete cds. |
| AA443993 | EST1088297 | 0.85 | FLJ20991 fis, clone |
| | | | CAE02103. |
| AA412533 | EST1056558 | 0.85 | Homo sapiens TIMM10 |
| | | | mRNA, complete cds. |
| AA393961 | EST1035294 | 0.84 | Homo sapiens cDNA |
| | | | FLJ20304 fis, clone |
| | | | HEP06749. |
| W90412 | unknown | 0.84 | unknown |
| N21209 | EST418492 | 0.82 | unknown |
| AA441897 | EST1086191 | 0.82 | Homo sapiens, eukaryotic |
| | | | translation initiation |
| | | | factor 4E binding protein |
| | | | 2, clone MGC: 12944, |
| | | | mRNA, complete cds. |
| AA454159 | EST1098658 | 0.82 | Homo sapiens putative |
| | | | cellular retinol-binding |
| | | | protein CRBP III mRNA, |
| | | | complete cds. |
| AA187151 | EST827313 | 0.82 | Homo sapiens LIM- |
| | | | homeodomain protein |
| | | | HLHX2 (LHX2) mRNA, |
| | | | complete cds. |
| AA460659 | EST1105394 | 0.82 | Homo sapiens, |
| | | | hypothetical protein |
| | | | FLJ20254, clone |
| | | | MGC: 3089, mRNA, |
| | | | complete cds. |
| AA465520 | EST1110046 | 0.81 | unknown |
| R49117 | EST227214 | 0.81 | H. sapiens HLA class III |
| | | | DNA. |
| R49126 | EST227222 | 0.81 | unknown |
| AA088276 | EST724635 | 0.81 | Mus musculus adult male |
| | | | urinary bladder cDNA, |
| | | | RIKEN full-length |
| | | | enriched library, |
| | | | clone: 9530018H14, full |
| | | | insert sequence. |
| N47589 | EST453118 | 0.81 | unknown |
| M80651 | HUMCEI5A | 0.81 | Hum ORF (CEI5) |
| | | | mRNA, 3′ flank. |
| AA449914 | EST1094493 | 0.81 | Homo sapiens y + L amino |
| | | | acid transporter-1 |
| | | | mRNA, complete cds. |
| AB002378 | AB002378 | 0.80 | Rattus norvegicus |
| | | | RhoGEF glutamate |
| | | | transport modulator |
| | | | GTRAP48 mRNA, |
| | | | complete cds. |
| AA435746.2 | unknown | 0.80 | unknown |
| AA456612 | EST1100967 | 0.80 | Homo sapiens HSPC262 |
| | | | mRNA, partial cds. |
| T47601 | unknown | −0.80 | unknown |
| R41329 | EST224800 | −0.80 | no description available |
| H82424 | EST384723 | −0.80 | Homo sapiens multi- |
| | | | PDZ-domain-containing |
| | | | protein mRNA, complete |
| | | | cds. |
| AA182001.2 | unknown | −0.80 | unknown |
| W01296 | EST508321 | −0.80 | Macaca fascicularis brain |
| | | | cDNA, clone: QnpA- |
| | | | 18080. |
| W26762 | EST532966 | −0.81 | FLJ21168 fis, clone |
| | | | CAS10874. |
| N95337 | EST502303 | −0.81 | unknown |
| R80332 | EST257268 | −0.81 | unknown |
| AA082041 | EST717271 | −0.81 | Homo sapiens cDNA |
| | | | FLJ13864 fis, clone |
| | | | THYRO1001173. |
| N21614 | EST418897 | −0.81 | Homo sapiens basic- |
| | | | leucine zipper |
| | | | transcription factor MafG |
| | | | (MAFG) mRNA, |
| | | | complete cds. |
| AA490262 | EST1134503 | −0.81 | Mus musculus, clone |
| | | | IMAGE: 3484373, |
| | | | mRNA, partial cds. |
| N24990 | EST423237 | −0.82 | Mus musculus p53- |
| | | | inducible zinc finger |
| | | | protein (Wig-1) mRNA, |
| | | | complete cds. |
| AA449973 | EST1094552 | −0.82 | Homo sapiens mRNA for |
| | | | cisplatin resistance- |
| | | | associated overexpressed |
| | | | protein, complete cds. |
| N34817 | EST436210 | −0.82 | Macaca fascicularis brain |
| | | | cDNA, clone: QnpA- |
| | | | 13493. |
| AA150205 | EST790106 | −0.82 | Homo sapiens cDNA |
| | | | FLJ10107 fis, clone |
| | | | HEMBA 1002583. |
| AA456048 | EST100603 | −0.82 | unknown |
| AA454930 | EST1099485 | −0.82 | FLJ23249 fis, clone |
| | | | COL04196. |
| AA291629 | EST932930 | −0.82 | Homo sapiens Crk- |
| | | | associated substrate |
| | | | p130Cas mRNA, |
| | | | complete cds. |
| AA443683 | EST1087987 | −0.82 | Homo sapiens CGI-113 |
| | | | protein mRNA, complete |
| | | | cds. |
| AA205334 | EST845853 | −0.83 | Homo sapiens mRNA; |
| | | | cDNA DKFZp566M063 |
| | | | (from clone |
| | | | DKFZp566M063). |
| C14823 | unknown | −0.83 | unknown |
| N69322 | EST475403 | −0.83 | H. sapiens mRNA for |
| | | | collagenase 3. |
| AA425724 | EST1069458 | −0.83 | Human R kappa B |
| | | | mRNA, complete cds. |
| AA425665 | EST1069399 | −0.84 | unknown |
| D60062 | EST335049 | −0.84 | X. laevis mRNA for |
| | | | XLCL2 protein. |
| D51060 | EST324948 | −0.85 | influenza virus |
| | | | hemagglutinin 5′ epitope |
| | | | tag = fusion protein {frame |
| | | | 1, multiple cloning site} |
| | | | [Saccharomyces |
| | | | cerevisiae = yeast, cloning |
| | | | vector YCpIF15, 16, 17, |
| | | | Other Plasmid Synthetic |
| | | | Partial, 135 nt]; fusion |
| | | | protein |
| AA496110 | EST1140740 | −0.85 | Mus musculus ES cells |
| | | | cDNA, RIKEN full- |
| | | | length enriched library, |
| | | | clone: 2410080H04, full |
| | | | insert sequence. |
| AB002346 | AB002346 | −0.85 | Homo sapiens |
| | | | synaptojanin 2 mRNA, |
| | | | complete cds. |
| H88359 | EST405813 | −0.85 | Nrf2 = NF-E2-like basic |
| | | | leucine zipper |
| | | | transcriptional activator |
| | | | [human, hemin-induced |
| | | | K562 cells, mRNA, 2304 |
| | | | nt]. |
| Z40074 | EST66200 | −0.85 | Homo sapiens cDNA |
| | | | FLJ10997 fis, clone |
| | | | PLACE1002438, weakly |
| | | | similar to ZINC FINGER |
| | | | PROTEIN 151. |
| AA426468 | EST1069728 | −0.85 | FLJ21313 fis, clone |
| | | | COL02176. |
| AA404500 | EST1046036 | −0.85 | Equus caballus mRNA |
| | | | for follistatin, complete |
| | | | cds. |
| AA485428 | EST1130058 | −0.85 | FLJ22735 fis, clone |
| | | | HUV00180. |
| AA282405 | EST923595 | −0.86 | Homo sapiens ubiquitous |
| | | | 6-phosphofructo-2- |
| | | | kinase/fructose 2,6- |
| | | | bisphosphatase |
| | | | (PFKFB3) mRNA, |
| | | | complete cds. |
| AA074514 | EST709727 | −0.86 | Rattus norvegicus mRNA |
| | | | for p65 protein. |
| R57419 | EST234156 | −0.86 | unknown |
| AA386265 | EST1027590 | −0.87 | unknown |
| AA384503 | EST1025832 | −0.87 | Homo sapiens |
| | | | polyglutamine-containing |
| | | | C14ORF4 gene. |
| AA419427 | EST1062073 | −0.87 | unknown |
| AA621004 | EST1318811 | −0.88 | unknown |
| AA065217 | EST690989 | −0.88 | Homo sapiens mRNA for |
| | | | Frizzled-6, complete cds. |
| AA279420 | EST920265 | −0.88 | Homo sapiens |
| | | | spliceosomal protein SAP |
| | | | 155 mRNA, complete |
| | | | cds. |
| AA037192 | EST652853 | −0.88 | Homo sapiens mRNA; |
| | | | cDNA DKFZp586O1919 |
| | | | (from clone |
| | | | DKFZp586O1919). |
| AA405205 | EST1046917 | −0.88 | H. sapiens mRNA for |
| | | | testican. |
| AA405559 | EST1046433 | −0.89 | unknown |
| AA398346 | EST1039764 | −0.89 | Homo sapiens cDNA |
| | | | FLJ11995 fis, clone |
| | | | HEMBB1001443, highly |
| | | | similar to Rattus |
| | | | norvegicus pyruvate |
| | | | dehydrogenase |
| | | | phosphatase isoenzyme 1 |
| | | | mRNA. |
| AA443328 | EST1087632 | −0.89 | Mus musculus adult male |
| | | | stomach cDNA, RIKEN |
| | | | full-length enriched |
| | | | library, |
| | | | clone: 2210410E06, full |
| | | | insert sequence. |
| H88706 | EST391015 | −0.90 | Homo sapiens full length |
| | | | insert cDNA clone |
| | | | YW23E08. |
| AA047616 | EST663260 | −0.91 | Homo sapiens cDNA |
| | | | FLJ11196 fis, clone |
| | | | PLACE1007688, weakly |
| | | | similar to LA PROTEIN |
| | | | HOMOLOG. |
| N40168 | unknown | −0.93 | unknown |
| AA253043 | EST891716 | −0.93 | FLJ21563 fis, clone |
| | | | COL06445. |

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polynucleotide that encodes a polypeptide comprising a sequence of amino acid residues that is at least 90% identical to an amino acid sequence selected from the group consisting of:
(a) the amino acid sequence as shown in SEQ ID NO:2 from amino acid number 32 (His), to amino acid number 253 (Phe); and
(b) the amino acid sequence as shown in SEQ ID NO:2 from amino acid number 1 (Met), to amino acid number 253 (Phe); and
(c) a polynucleotide sequence complementary to (a) or (b).

2. An isolated polynucleotide according to claim 1, wherein the polynucleotide is selected from the group consisting of:
(a) a polynucleotide sequence as shown in SEQ ID NO:1 from nucleotide 298 to nucleotide 962;
(b) a polynucleotide sequence as shown in SEQ ID NO:1 from nucleotide 205 to nucleotide 962; and
(c) a polynucleotide sequence complementary to (a) or (b).

3. An isolated polynucleotide sequence according to claim 1, wherein the polynucleotide comprises nucleotide 94 to nucleotide 759 of SEQ ID NO:5.

4. An isolated polynucleotide according to claim 1, wherein the polypeptide comprises a sequence of amino acid residues selected from the group consisting of:
(a) the amino acid sequence as shown in SEQ ID NO:2 from amino acid number 32 (His), to amino acid number 253 (Phe); and
(b) the amino acid sequence as shown in SEQ ID NO:2 from amino acid number 1 (Met), to amino acid number 253 (Phe); and
(c) a polynucleotide sequence complementary to (a) or (b).

5. An expression vector comprising the following operably linked elements:
a transcription promoter;
a DNA segment encoding a polypeptide as shown in SEQ ID NO:2 from amino acid number 32 (His), to amino acid number 253 (Phe); and
a transcription terminator,
wherein the promoter is operably linked to the DNA segment, and the DNA segment is operably linked to the transcription terminator.

6. An expression vector according to claim 5, further comprising a secretory signal sequence operably linked to the DNA segment.

7. A cultured cell comprising an expression vector according to claim 5, wherein the cell expresses a polypeptide encoded by the DNA segment.

8. A DNA construct encoding a fusion protein, the DNA construct comprising:
a first DNA segment encoding a polypeptide comprising a sequence of amino acid residues selected from the group consisting of:
(a) the amino acid sequence as shown in SEQ ID NO:2 from amino acid number 1 (Met), to amino acid number 31 (Leu);
(b) the amino acid sequence as shown in SEQ ID NO:4 from amino acid number 1 (Met), to amino acid number 27 (Arg);
(c) the amino acid sequence as shown in SEQ ID NO:2 from amino acid number 42 (Leu), to amino acid number 56 (Ile);
(d) the amino acid sequence as shown in SEQ ID NO:2 from amino acid number 108 (Tyr), to amino acid number 122 (Thr);
(e) the amino acid sequence as shown in SEQ ID NO:2 from amino acid number 151 (Ile), to amino acid number 165 (Gln);
(f) the amino acid sequence as shown in SEQ ID NO:2 from amino acid number 213 (Ile), to amino acid number 227 (Ala);
(g) the amino acid sequence as shown in SEQ ID NO:2 from amino acid number 42 (Ile), to amino acid number 227 (Ala);
(h) the amino acid sequence as shown in SEQ ID NO:2 from amino acid number 32 (His), to amino acid number 253 (Phe); and
at least one other DNA segment encoding an additional polypeptide,
wherein the first and other DNA segments are connected in-frame; and
wherein the first and other DNA segments encode the fusion protein.

9. An expression vector comprising the following operably linked elements:
a transcription promoter;
a DNA construct encoding a fusion protein according to claim 8; and
a transcription terminator,
wherein the promoter is operably linked to the DNA construct, and the DNA construct is operably linked to the transcription terminator.

10. A cultured cell comprising an expression vector according to claim 9, wherein the cell expresses a polypeptide encoded by the DNA construct.

11. A method of producing a fusion protein comprising:
culturing a cell according to claim 10; and
isolating the polypeptide produced by the cell.

12. An isolated polypeptide comprising a sequence of amino acid residues that is at least 90% identical to an amino acid sequence selected from the group consisting of:
(a) the amino acid sequence as shown in SEQ ID NO:2 from amino acid number 32 (His), to amino acid number 253 (Phe); and
(b) the amino acid sequence as shown in SEQ ID NO:2 from amino acid number 1 (Met), to amino acid number 253 (Phe).

13. An isolated polypeptide according to claim 12, wherein the polypeptide comprises a sequence of amino acid residues selected from the group consisting of:
(a) the amino acid sequence as shown in SEQ ID NO:2 from amino acid number 32 (His), to amino acid number 253 (Phe); and
(b) the amino acid sequence as shown in SEQ ID NO:2 from amino acid number 1 (Met), to amino acid number 253 (Phe).

14. A method of producing a polypeptide comprising:
culturing a cell according to claim 7; and
isolating the polypeptide produced by the cell.

15. A method of producing an antibody comprising:
inoculating an animal with a polypeptide selected from the group consisting of:
(a) a polypeptide according to claim 13;
(b) a polypeptide comprising the amino acid sequence of SEQ ID NO:2 from amino acid number 42 (Leu), to amino acid number 56 (Ile);
(c) a polypeptide comprising the amino acid sequence of SEQ ID NO:2 from amino acid number 108 (Tyr), to amino acid number 122 (Thr);
(d) a polypeptide comprising the amino acid sequence of SEQ ID NO:2 from amino acid number 151 (Ile), to amino acid number 165 (Gln);
(e) a polypeptide comprising the amino acid sequence of SEQ ID NO:2 from amino acid number 213 (Ile), to amino acid number 227 (Ala);
(f) a polypeptide comprising the amino acid sequence of SEQ ID NO:2 from amino acid number 42 (Ile), to amino acid number 227 (Ala);
(g) a polypeptide comprising the amino acid sequence of SEQ ID NO:2 from amino acid number 34 (Gln) to amino acid number 39 (Arg);
(h) a polypeptide comprising the amino acid sequence of SEQ ID NO:2 from amino acid number 59 (Asn) to amino acid number 64 (Asp);
(i) a polypeptide comprising the amino acid sequence of SEQ ID NO:2 from amino acid number 63 (Lys) of SEQ ID NO:2; and (4) amino acid number 116 (Gly);
(j) a polypeptide comprising the amino acid sequence of SEQ ID NO:2 from amino acid number 58 (Ala) to amino acid number 121 (Glu);
(k) a polypeptide comprising the amino acid sequence of SEQ ID NO:2 from amino acid number 101 (Glu) to amino acid number 107 (Leu);
(l) a polypeptide comprising the amino acid sequence of SEQ ID NO:2 from amino acid number 162 (Thr) to amino acid number 169 (Glu);
(m) a polypeptide comprising the amino acid sequence of SEQ ID NO:2 from amino acid number 194 (Lys) to amino acid number 200 (Leu);
(n) a polypeptide comprising the amino acid sequence of SEQ ID NO:2 from amino acid number 218 (Cys) to amino acid number 225 (Asp);
(o) a polypeptide comprising the amino acid sequence of SEQ ID NO:2 from amino acid number Ala (249) to amino acid number 252 (Arg); and
wherein the polypeptide elicits an immune response in the animal to produce the antibody; and
isolating the antibody from the animal.

16. An antibody produced by the method of claim 15, which binds to a polypeptide as shown in SEQ ID NO:2 from amino acid 32-353.

17. The antibody of claim 16, wherein the antibody is a monoclonal antibody.

18. An antibody that specifically binds to a polypeptide of claim 13.

19. A method of detecting, in a test sample, the presence of an antagonist of zlmda24 protein activity, comprising:
culturing a cell that is responsive to a zlmda24-stimulated cellular pathway; and
producing a zlmda24 polypeptide by the method of claim 14; and
exposing the zlmda24 polypeptide to the cell, in the presence and absence of a test sample; and
comparing levels of response to the zlmda24 polypeptide, in the presence and absence of the test sample, by a biological or biochemical assay; and
determining from the comparison, the presence of the antagonist of zlmda24 activity in the test sample.

20. A method of detecting, in a test sample, the presence of an agonist of zlmda24 protein activity, comprising:
culturing a cell that is responsive to a zlmda24-stimulated cellular pathway; and
adding a test sample; and
comparing levels of response in the presence and absence of the test sample, by a biological or biochemical assay; and
determining from the comparison, the presence of the agonist of zlmda24 activity in the test sample.

21. A method for detecting a genetic abnormality in a patient, comprising:
obtaining a genetic sample from a patient;
producing a first reaction product by incubating the genetic sample with a polynucleotide comprising at least 14 contiguous nucleotides of SEQ ID NO:1 or the complement of SEQ ID NO:1, under conditions wherein said polynucleotide will hybridize to complementary polynucleotide sequence;
visualizing the first reaction product; and comparing said first reaction product to a control reaction product from a wild type patient, wherein a difference between said first reaction product and said control reaction product is indicative of a genetic abnormality in the patient.

22. A method for detecting testis tissue in a patient sample, comprising:
obtaining a tissue or biological sample from a patient;
incubating the tissue or biological sample with an antibody of claim 18 under conditions wherein the antibody binds to its complementary polypeptide in the tissue or biological sample;
visualizing the antibody bound in the tissue or biological sample; and
comparing levels and localization of antibody bound in the tissue or biological sample from the patient to a non-testis control tissue or biological sample,
wherein an increase in the level or localization of antibody bound to the patient tissue or biological sample relative to the non-testis control tissue or biological sample is indicative of testis tissue in a patient sample.

23. A method for detecting a testicular cancer in a patient, comprising:
obtaining a tissue or biological sample from a patient;
incubating the tissue or biological sample with an antibody of claim 18 under conditions wherein the antibody binds to its complementary polypeptide in the tissue or biological sample;
visualizing the antibody bound in the tissue or biological sample; and
comparing levels of antibody bound in the tissue or biological sample from the patient to a normal control tissue or biological sample,
wherein an increase in the level of antibody bound to the patient tissue or biological sample relative to the normal control tissue or biological sample is indicative of a testicular cancer in the patient.

24. A method for detecting testis tissue in a patient sample, comprising:
obtaining a tissue or biological sample from a patient;
labeling a polynucleotide comprising at least 14 contiguous nucleotides of SEQ ID NO:1 or the complement of SEQ ID NO:1;
incubating the tissue or biological sample with under conditions wherein the polynucleotide will hybridize to complementary polynucleotide sequence;
visualizing the labeled polynucleotide in the tissue or biological sample; and
comparing the level and localization of labeled polynucleotide hybridization in the tissue or biological sample from the patient to a control non-testis tissue or biological sample,
wherein an increase in the level or localization of the labeled polynucleotide hybridization to the patient tissue or biological sample relative to the control non-testis tissue or biological sample is indicative of testis tissue in a patient sample.

25. A method for detecting a testicular cancer in a patient, comprising:
obtaining a tissue or biological sample from a patient;
labeling a polynucleotide comprising at least 14 contiguous nucleotides of SEQ ID NO:1 or the complement of SEQ ID NO:1;
incubating the tissue or biological sample with under conditions wherein the polynucleotide will hybridize to complementary polynucleotide sequence;
visualizing the labeled polynucleotide in the tissue or biological sample; and
comparing the level of labeled polynucleotide hybridization in the tissue or biological sample from the patient to a normal control tissue or biological sample,
wherein an increase in the labeled polynucleotide hybridization to the patient tissue or biological sample relative to the normal control tissue or biological sample is indicative of a testicular cancer in the patient.


SEQUENCE LISTING (25 sequences)

SEQ ID NO: 1 - DNA, 1041 bp, Homo sapiens
  CDS (205)...(966)
ctgagaacaccagaggactggcagttgggagcctgatggaggacaagtagggcctcgaggacaggtgcgtgacagaagcacaggaaaaaaaagaaaaatgaagaaataaaaacacgagttcatcagtaaagaggtaccctggcagcataaatattatgataagctaaaagctggaatcatctggaaaaataaataagactcctcatgtccttttcggtccataaccagaagggcagcaaaaggcctttgccactggaacctcttctttttctccaagtcccacgtagcaattacctgcactttcaagaagagaaacaacgactacacctaaagaaattccttcttgataggatgtttctagtggccaagatacaagcaaatgtagaaagaaaagatgttgctgactactatgaacaaatgtttcagtcagttttgaaacatcacctaggagaagcagtgacaggattgctgctcatctatcccacttccattctgcatatcctcgagtcctccagcgacactctctacaaagttcttttagattatattggccatgtcaaagatgaaacagtattttttattcaacaaatgaaaattatagtcatttctcataacattccaatgaggctttttatgcaatggcatgtttcagtgataaaagttccagttatgtatctcgacgatgtgacacagtcacagtccctaaaggaggtcatcacagattttctcacacaaactcataaactgtcactctacctttgccagactatgaaagtaggcactaaaggaccaggcgataacttacaccaagttgcacctgacctactcctcccagaacaaatcataaagtacttgtgcaaatccgaagaattcatggacccggcaacatttataaacatgtataatagacccatacacatcactctggattctgaggtggtatggcctgctccttcacgtttctaggattgagagggataatgtgcccatgtctcttaaggagtttgtgctacttaaataaaaaaaacatttttaaagtta

SEQ ID NO: 2 - protein, 253 aa, Human
MSFSVHNQKGSKRPLPLEPLLFLQVPRSNYLHFQEEKQRLHLKKFLLDRMFLVAKIQANVERKDVADYYEQMFQSVLKHHLGEAVTGLLLIYPTSILHILESSSDTLYKVLLDYIGHVKDETVFFIQQMKIIVISHNIPMRLFMQWHVSVIKVPVMYLDDVTQSQSLKEVITDFLTQTHKLSLYLCQTMKVGTKGPGDNLHQVAPDLLLPEQIIKYLCKSEEFMDPATFINMYNRPIHITLDSEVVWPAPSRF

SEQ ID NO: 3 - DNA, 1056 bp, Mus musculus
  CDS (106)...(867)
gcagaggtcaagccaagaggacctcagacctcaaagacagaaataaaaataagaattcactaccattgagacttgtaatcatctaaaaaagaaataagattcatcatgtctttcatggtccataatcggaagggcagcaaaaagcaatttcaagtggatcctcttcttctgcccaaggttcctcgtaccaattacctgcaccttcaggaagagaagcacagactacagctaaagaaattcctccttcacaggatgtttctagtgggctacatacaaggcaacacggagaaaaaggacatctctgagtactatgagcaactgtttcagtcaattctgaaacatcatttaggcgaatcagtgacaggtctcatgctcgtatacccaagtacttttctgcacattcttgagagttccaatggcacactcttccggattcttctagattatgttgcccatgaaaagagtgaaacagaatttatgctccaaaacatgaaaatcgttgttgcttctcacaacatccccacgaggctgttcatgcagtggcatatctctgcaatcaaagtccccgttttgtacctagatgacgaatcacagtctccgtctatagaggaagtcaccacagaattcctcaccatgactcacaaactggcactccaactttacaagacagtgaaattgggcgcgaaaggtccaggtgacaacttacaccaacttgcccctgaactcattctcccagaacaaattatcaagtatttatgcaaagctgaagaattcatggacccagcgtctttcttgagcatgtataacagacccatacacgttaccctggattccgatattgtgtggccagctccttcccgtttctagaatggggagagttgatttggagaggatgtctacaacattcagaagcattgaagatggcaccagagcaggagggtgaaagaacgcatgttcagccaggcatagaacaggagccaggaaggggacaacaccataaattcctgaatttctttgtgttttaggcactaaacataataaatttcttaagactga

SEQ ID NO: 4 - protein, 253 aa, Mus musculus
MSFMVHNRKGSKKQFQVDPLLLPKVPRTNYLHLQEEKHRLQLKKFLLHRMFLVGYIQGNTEKKDISEYYEQLFQSILKHHLGESVTGLMLVYPSTFLHILESSNGTLFRILLDYVAHEKSETEFMLQNMKIVVASHNIPTRLFMQWHISAIKVPVLYLDDESQSPSIEEVTTEFLTMTHKLALQLYKTVKLGAKGPGDNLHQLAPELILPEQIIKYLCKAEEFMDPASFLSMYNRPIHVTLDSDIVWPAPSRF

SEQ ID NO: 5 - DNA, 759 bp, Artificial Sequence
  Degenerate polynuclotide sequence of human zlmda24 shown in SEQ ID NO2
atgwsnttywsngtncayaaycaraarggnwsnaarmgnccnytnccnytngarccnytnytnttyytncargtnccnmgnwsnaaytayytncayttycargargaraarcarmgnytncayytnaaraarttyytnytngaymgnatgttyytngtngcnaarathcargcnaaygtngarmgnaargaygtngcngaytaytaygarcaratgttycarwsngtnytnaarcaycayytnggngargcngtnacnggnytnytnytnathtayccnacnwsnathytncayathytngarwsnwsnwsngayacnytntayaargtnytnytngaytayathggncaygtnaargaygaracngtnttyttyathcarcaratgaarathathgtnathwsncayaayathccnatgmgnytnttyatgcartggcaygtnwsngtnathaargtnwsngtnatgtayytngaygaygtnacncarwsncarwsnytnaargargtnathacngayttyytnacncaracncayaarytnwsnytntayytntgycaracnatgaargtnggnacnaarggnccnggngayaayytncaycargtngcnccngayytnytnytnccngarcarathathaartayytntgyaarwsngargarttyatggayccngcnacnttyathaayatgtayaaymgnccnathcayathacnytngaywsngargtngtntggccngcnccnwsnmgntty

SEQ ID NO: 6 - DNA, 759 bp, Artificial Sequence
  Degenerate polynucleotide sequence of mouse zlmda24 shown in SEQ ID NO4
atgwsnttyatggtncayaaymgnaarggnwsnaaraarcarttycargtngayccnytnytnytnccnaargtnccnmgnacnaaytayytncayytncargargaraarcaymgnytncarytnaaraarttyytnytncaymgnatgttyytngtnggntayathcarggnaayacngaraaraargayathwsngartaytaygarcarytnttycarwsnathytnaarcaycayytnggngarwsngtnacnggnytnatgytngtntayccnwsnacnttyytncayathytngarwsnwsnaayggnacnytnttymgnathytnytngaytaygtngcncaygaraarwsngaracngarttyatgytncaraayatgaarathgtngtngcnwsncayaayathccnacnmgnytnttyatgcartggcayathwsngcnathaargtnccngtnytntayytngaygaygarwsncarwsnccnwsnathgargargtnacnacngarttyytnacnatgacncayaarytngcnytncarytntayaaracngtnaarytnggngcnaarggnccnggngayaayytncaycarytngcnccngarytnathytnccngarcarathathaartayytntgyaargcngargarttyatggayccngcnwsnttyytnwsnatgtayaaymgnccnathcaygtnacnytngaywsngayathgtntggccngcnccnwsnmgntty

SEQ ID NO: 7 - DNA, 25 bp, Artificial Sequence
  Oligonucleotide primer ZC29714
aacagacccatacacgttaccctgg

SEQ ID NO: 8 - DNA, 23 bp, Artificial Sequence
  Oligonucleotide primer ZC29984
tcttgtccccttcctggctcctg

SEQ ID NO: 9 - DNA, 23 bp, Artificial Sequence
  Oligonucleotide primer ZC21195
gaggagaccataacccccgacag

SEQ ID NO: 10 - DNA, 23 bp, Artificial Sequence
  Oligonucleotide primer ZC21196
catagctcccaccacacgatttt

SEQ ID NO: 11 - DNA, 25 bp, Artificial Sequence
  Oligonucleotide primer ZC14063
caccagacataatagctgacagact

SEQ ID NO: 12 - DNA, 21 bp, Artificial Sequence
  Oligonucleotide primer ZC17574
ggtrttgctcagcatgcacac

SEQ ID NO: 13 - DNA, 24 bp, Artificial Sequence
  Oligonucleotide primer ZC17600
catgtaggccatgaggtccaccac

SEQ ID NO: 14 - DNA, 24 bp, Artificial Sequence
  Oligonucleotide primer ZC28587
gattgctgctcatctatcccactt

SEQ ID NO: 15 - DNA, 23 bp, Artificial Sequence
  Oligonucleotide primer ZC28591
tggtgtaagttatcgcctggtcc

SEQ ID NO: 16 - DNA, 18 bp, Artificial Sequence
  Oligonucleotide primer ZC29567
cctcccagaacaaatcat

SEQ ID NO: 17 - DNA, 18 bp, Artificial Sequence
  Oligonucleotide primer ZC29568
accacctcagaatccaga

SEQ ID NO: 18 - protein, 6 aa, Artificial Sequence
  Glu-Glu (CEE) Tag amino acid sequence
EYMPME

SEQ ID NO: 19 - DNA, 31 bp, Artificial Sequence
  Oligonucleotide Sequence ZC29055
cgcggatcccgatgtccttttcggtccataa

SEQ ID NO: 20 - DNA, 34 bp, Artificial Sequence
  Oligonucleotide Sequence ZC29056
tgctctagagcgaaacgtgaaggagcaggccata

SEQ ID NO: 21 - DNA, 17 bp, Artificial Sequence
  Oligonucleotide Sequence ZC447
taacaatttcacacagg

SEQ ID NO: 22 - DNA, 18 bp, Artificial Sequence
  Oligonucleotide Sequence ZC976
cgttgtaaaacgacggcc

SEQ ID NO: 23 - protein, 261 aa, Artificial Sequence [sequence omitted]

SEQ ID NO: 24 - DNA, 32 bp, Artificial Sequence
  Oligonucleotide Sequence ZC29127
cgtatcggccggccaccatgtccttttcggtc

SEQ ID NO: 25 - DNA, 32 bp, Artificial Sequence
  Oligonucleotide Sequence ZC29128
cgtatcggcgcgccctagaaacgtgaaggagc